(19) United States
(12) Patent Application Publication
(10) Pub. No.: US 2024/0002442 A1
(43) Pub. Date: Jan. 4, 2024

(54) CYCLIC PEPTIDES WITH ANTIMICROBIAL PROPERTIES

(71) Applicant: Northeastern University, Boston, MA (US)

(72) Inventors: Kim Lewis, Newton, MA (US); Ryan Miller, Greenville, RI (US); Akira Iinishi, Newton, MA (US); Patrick James Lariviere, Cambridge, MA (US); Thomas Curtis, Boston, MA (US)

(21) Appl. No.: 18/254,459

(22) PCT Filed: Nov. 24, 2021
(86) PCT No.: PCT/US2021/060837

§ 371 Date: May 25, 2023

Related U.S. Application Data
(60) Provisional application No. 63/118,254, filed on Nov. 25, 2020.
(60) Provisional application No. 63/172,163, filed on Apr. 8, 2021.

(51) Int. Cl. C07K 7/64; A01N 63/50; A01P 1/00

(52) U.S. Cl. CPC C07K 7/64; A01N 63/50; A01P 1/00; A61K 38/00

(57) ABSTRACT

Novel compounds and analogues that exhibit antimicrobial activity—particularly against Gram-negative pathogens—are producible from bacterial isolate Photorhabdus australis DSM 17609 and some related bacterial species of the genus. Pharmaceutical compositions containing the novel compound and its analogues are useful for treating or preventing a bacterial infection. Compounds in accordance herewith include ribosomally produced and post-translationally modified cyclic peptides useful for the treatment, amelioration, and prevention of bacterial infections by Gram-negative pathogens, in addition to other indications.

### CROSS-REFERENCE TO RELATED APPLICATION(S)

This application claims the benefit of and priority to U.S. Provisional Application No. 63/118,254, filed on Nov. 25, 2020, and 63/172,163, filed on Apr. 8, 2021, which are incorporated herein by reference in their entireties.

### GOVERNMENT RIGHTS

This invention was made with government support under Grant No. P01-AI118687 awarded by the National Institutes of Health. The government has certain rights in the invention.

### FIELD OF THE INVENTION

The present disclosure relates to a novel antimicrobial compound and its analogues, pharmaceutical compositions comprising said compounds and the use of said compounds and pharmaceutical compositions for treatment. This invention was made with government support under Grant No. P01-All 18687 awarded by the National Institutes of Health. The government has certain rights in the invention.

### BACKGROUND OF THE INVENTION

Infectious diseases without useful therapeutics represent perhaps the largest public health threat of our time, recently highlighted by a global pandemic. The successes of modern medicine are owed to the availability of safe and effective antimicrobial agents, and the growing severity of threats to health is inexorably linked to the lack of novel leads in drug-discovery pipelines. Reports of multidrug-resistant pathogens continue to rise.

In susceptible individuals, certain Gram-negative bacteria can cause serious complications and infections, such as pneumonia, urinary tract infections, wound infections, ear infections, eye infections, intra-abdominal infections, oral bacterial overgrowth and sepsis. The treatment of serious bacterial infections in clinical practice can be complicated by antibiotic resistance. Recent years have seen a rise in infections by Gram-negative bacteria that are resistant to many types of antimicrobials, including broad-spectrum antibiotics such as aminoglycosides, cephalosporins, and even carbapenems. Gram-negative bacteria render most antibiotics ineffective by their near-impermeable outer membrane barrier and sophisticated efflux mechanisms. The rules mediating drug penetration and accumulation in Gram-negative bacteria are poorly understood, and may explain why rational drug design has failed to produce synthetic leads, underscored by the accidental discovery in 1968 of quinolones—the last novel class of broad spectrum antibiotics to enter clinical use.

Selective activity against Gram-negative pathogens is largely limited to the class of drugs known as polymyxins, which are produced by Paenibacillus species; however, these have the large drawback of significant nephrotoxicity and neurotoxicity, and are usually held in reserve as a last line of defense against multidrug-resistant Gram-negative pathogens.

### SUMMARY OF THE INVENTION

The present invention pertains to novel compounds and analogues that exhibit antimicrobial activity—particularly against Gram-negative pathogens. Pharmaceutical compositions containing the novel compound and its analogues are useful for treating or preventing a bacterial infection. Compounds in accordance herewith include ribosomally produced and post-translationally modified cyclic peptides useful for the treatment, amelioration, and prevention of bacterial infections by Gram-negative pathogens, in addition to other indications. Embodiments of the invention include therapeutically useful analogs of these compounds and pharmaceutical compositions containing the compounds of the invention for the treatment, amelioration, or prevention of various infectious diseases.

In one aspect, the present invention comprises, consists essentially of or consists of a novel compound represented by one or more of the following Formulae (I)-(XIII):

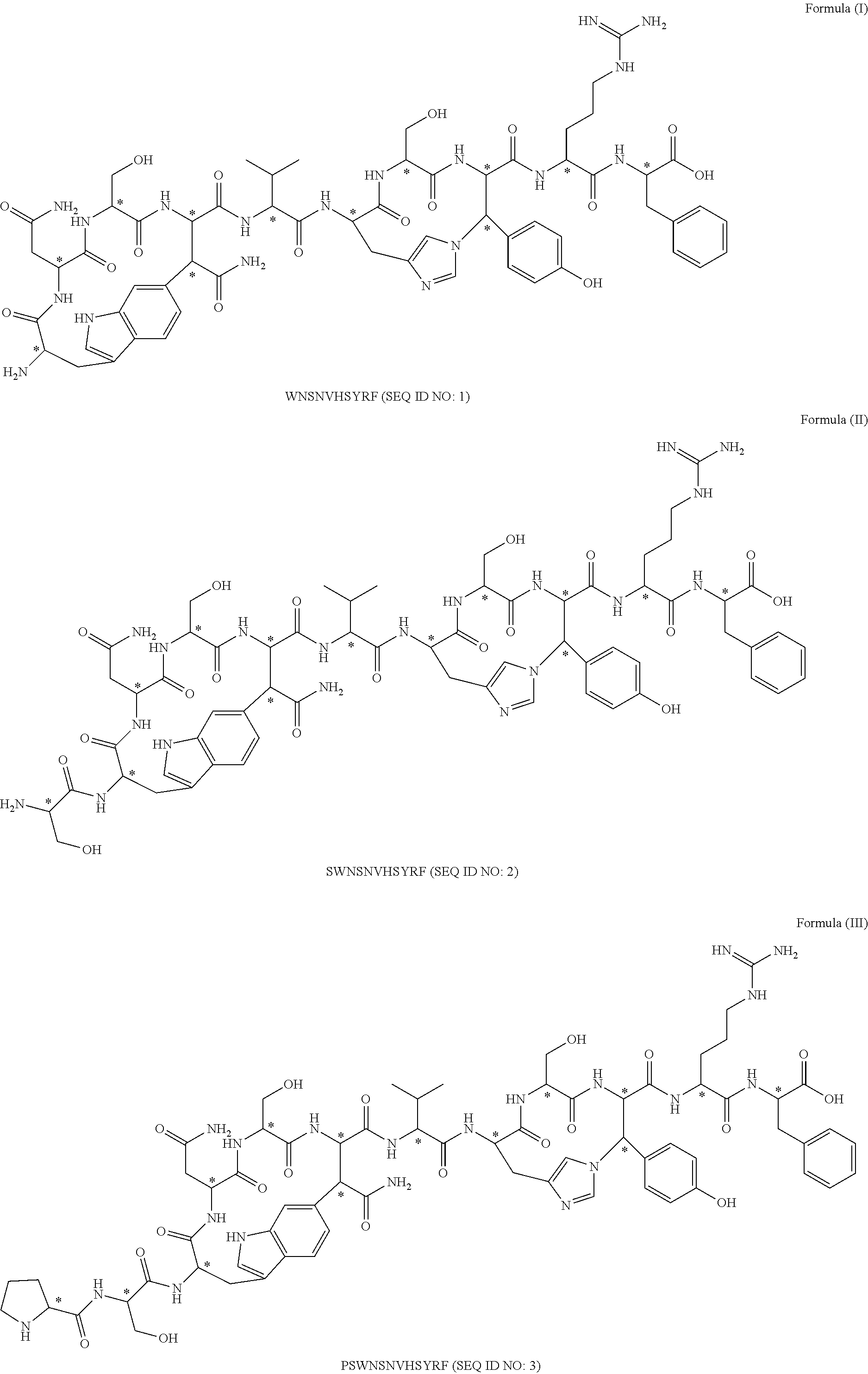

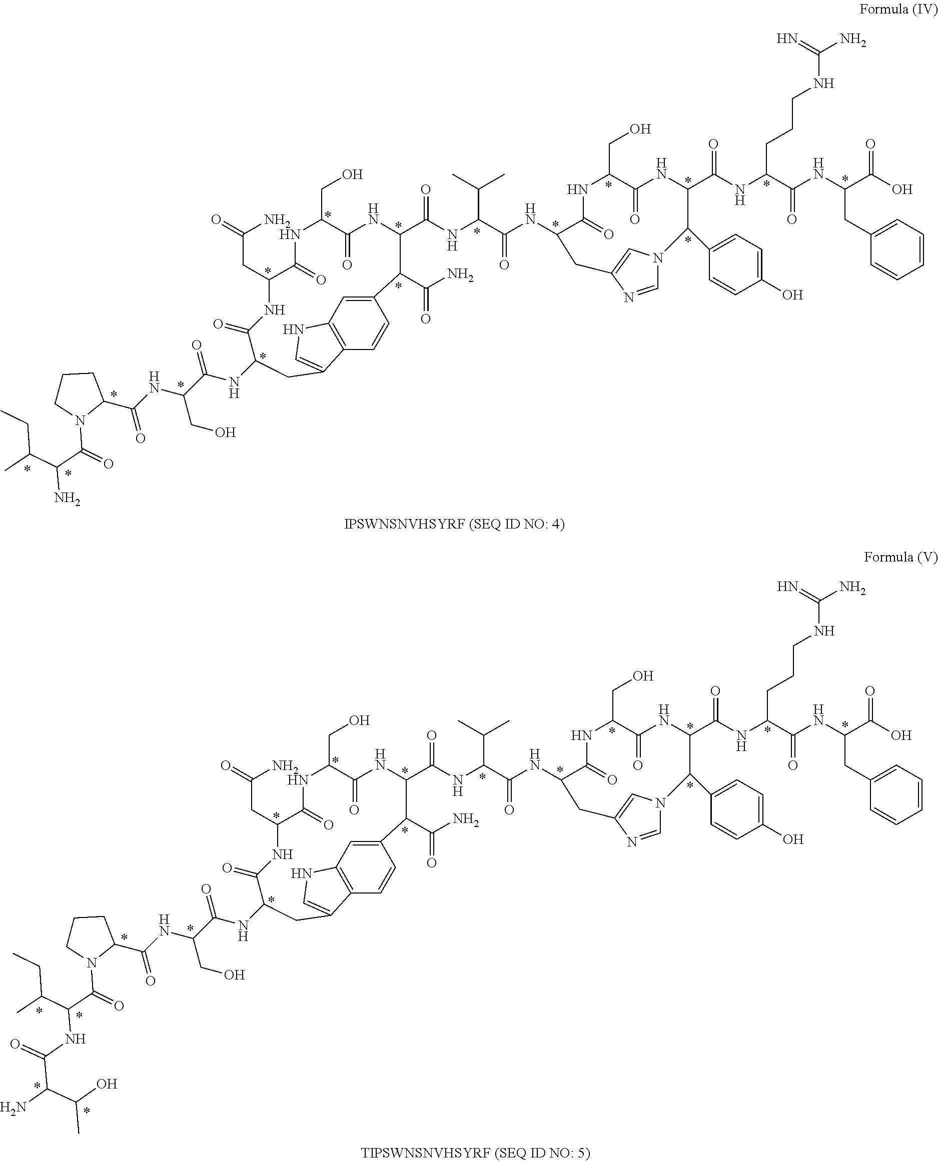 [SMILES: CCC(C)C(N)C(=O)N1CCCC1C(=O)NC(CO)C(=O)NC1Cc2c[nH]c3cc(ccc23)C(C(N)=O)C(C(=O)NC(C(=O)NC2Cc3cn(cn3)C(c3ccc(O)cc3)C(C(=O)NC(CCCNC(=N)N)C(=O)NC(Cc3ccccc3)C(=O)O)NC(=O)C(CO)NC2=O)C(C)C)NC(=O)C(CO)NC(=O)C(CC(N)=O)NC1=O ; CCC(C)C(NC(=O)C(N)C(C)O)C(=O)N1CCCC1C(=O)NC(CO)C(=O)NC1Cc2c[nH]c3cc(ccc23)C(C(N)=O)C(C(=O)NC(C(=O)NC2Cc3cn(cn3)C(c3ccc(O)cc3)C(C(=O)NC(CCCNC(=N)N)C(=O)NC(Cc3ccccc3)C(=O)O)NC(=O)C(CO)NC2=O)C(C)C)NC(=O)C(CO)NC(=O)C(CC(N)=O)NC1=O]

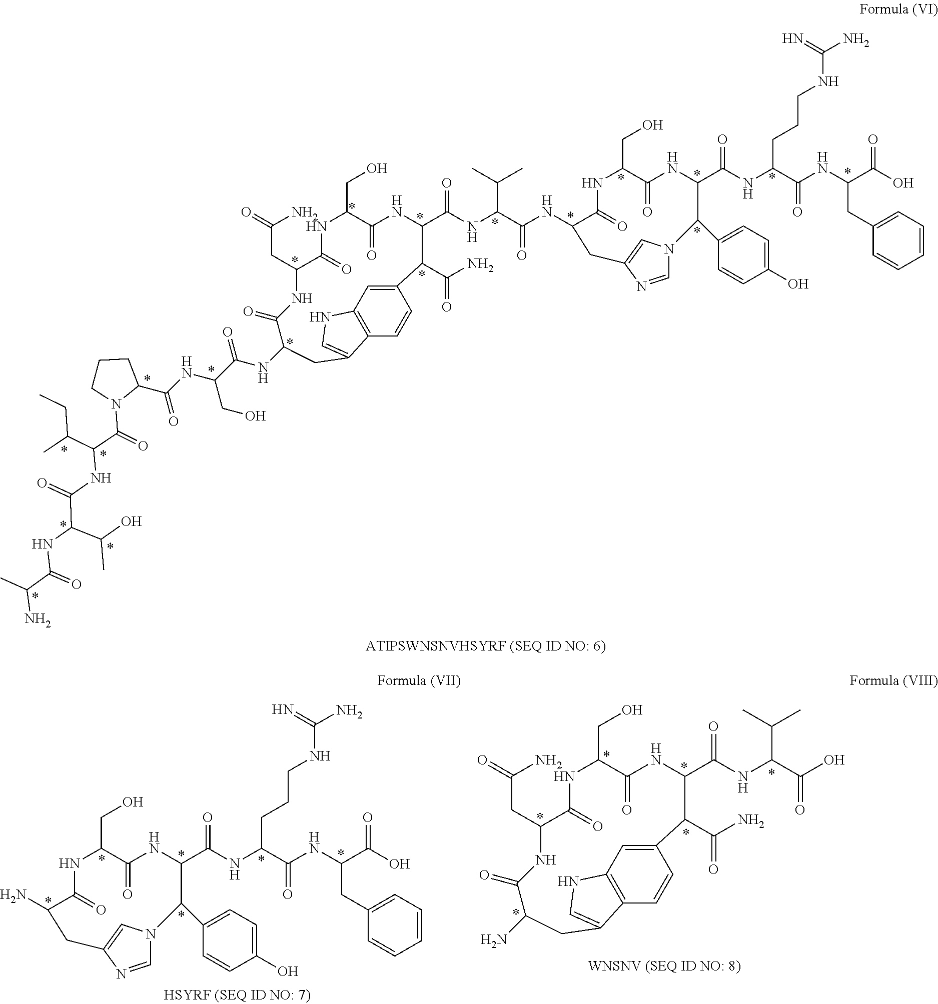 [SMILES: CCC(C)C(NC(=O)C(NC(=O)C(C)N)C(C)O)C(=O)N1CCCC1C(=O)NC(CO)C(=O)NC1Cc2c[nH]c3cc(ccc23)C(C(N)=O)C(C(=O)NC(C(=O)NC2Cc3cn(cn3)C(c3ccc(O)cc3)C(C(=O)NC(CCCNC(=N)N)C(=O)NC(Cc3ccccc3)C(=O)O)NC(=O)C(CO)NC2=O)C(C)C)NC(=O)C(CO)NC(=O)C(CC(N)=O)NC1=O ; N=C(N)NCCCC(NC(=O)C1NC(=O)C(CO)NC(=O)C(N)Cc2cn(cn2)C1c1ccc(O)cc1)C(=O)NC(Cc1ccccc1)C(=O)O ; CC(C)C(NC(=O)C1NC(=O)C(CO)NC(=O)C(CC(N)=O)NC(=O)C(N)Cc2c[nH]c3cc(ccc23)C1C(N)=O)C(=O)O]

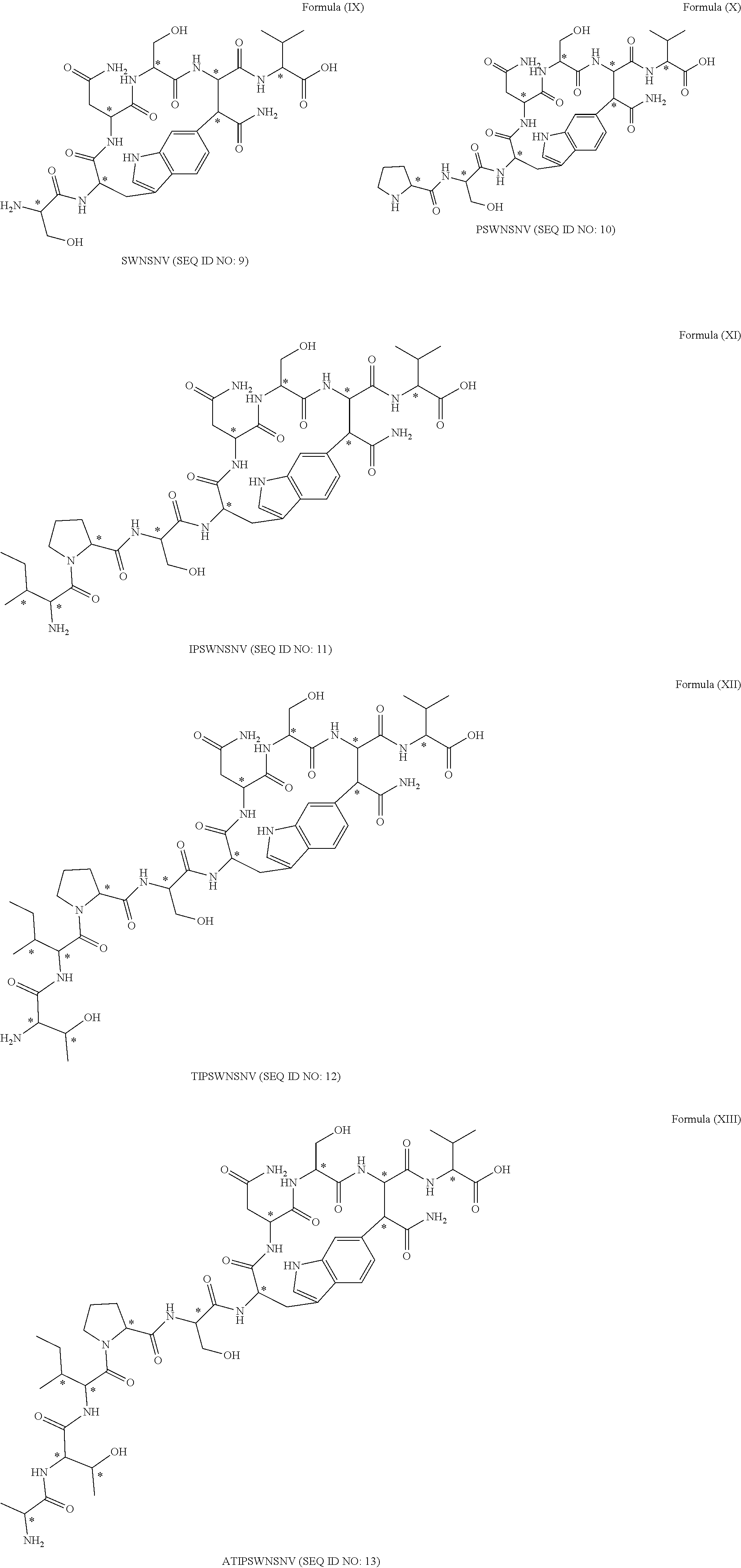

and/or an enantiomer, diastereomer, tautomer, rotamer, racemates, prodrugs, hydrates, or pharmaceutically acceptable salts thereof, wherein stereocenters can be either R or S configuration (stereocenters indicated by asterisk “*”). The invention includes pharmaceutically acceptable salts, stereoisomers (including enantiomers), tautomers, or hydrates thereof, as well as analogues of Formulae (I)-(XIII) embodied by Schemes (I)-(IV). The present invention also includes pharmaceutical compositions comprising or consisting essentially of any one or more of Formula (I)-(XIII) compounds or their analogues, the use of any one or more of Formula (I)-(XIII) compounds and their analogues, and methods for treating or preventing a bacterial infection with any one or more of Formula (I)-(XIII) compounds or analogues thereof.

Chemical analysis of compounds of Formulae (I)-(VI) appear in FIGS. 5-8. In some embodiments, Formulae (I)-(XIII) or analogues thereof are natural products isolated from bacterial species. For example, in some embodiments, isolated natural products corresponding to one or more of Formulae (I)-(XIII) are produced by bacterial isolate Photorhabdus australis strain DSM 17609 and related members of the bacterial species whose genome contains the biosynthetic cluster described below. For example, the compounds of Formulae (I)-(XIII) are producible from bacterial isolate Photorhabdus australis DSM 17609 and some related bacterial species of the genus.

In another aspect, the present invention relates to a pharmaceutical composition comprising one or more compounds in accordance with any one or more of Formulae (I)-(XIII) and a pharmaceutically acceptable excipient, carrier, surfactant, liquid and/or solid diluent. In some embodiments, the pharmaceutical composition may further include an agent selected from the group consisting of an antibiotic, antifungal, antiviral, anti-protozoal, anthelminthic, anti-neoplastic, immune-regulatory, anti-hypercholesterolemia agents, and combinations thereof.

In still another aspect, the present invention pertains to a method for producing any of the compounds of Formulae (I)-(XIII), analogues embodied by Scheme (I)-(IV), or an enantiomer, diastereomer, tautomer, rotamer, racemates, prodrug, hydrate, or pharmaceutically acceptable salt thereof. In various embodiments, the method comprises cultivating a bacterial isolate, Photorhabdus australis DSM 17609 for example, in a culture comprising resources of carbon, nitrogen, and inorganic salts under aerobic conditions, thereby producing one or more compounds in accordance with Formulae (I)-(XIII). In some embodiments, the method further includes the isolation of these compounds.

An additional aspect of the invention is that the present invention comprises, consists essentially of or consists of novel compounds represented by one or more of the following generalized formulae embodied by Schemes (I)-(IV):

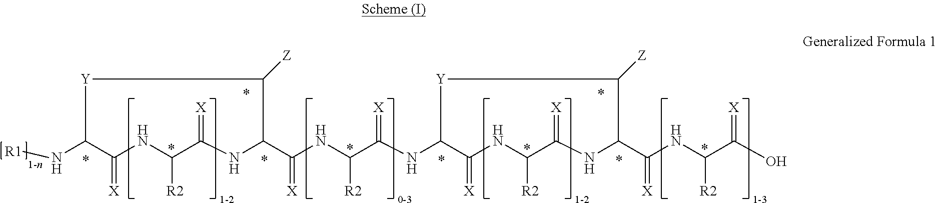

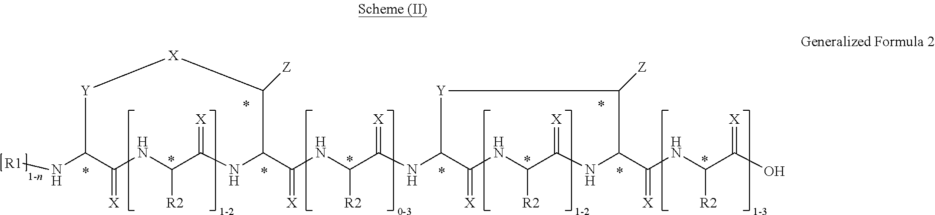

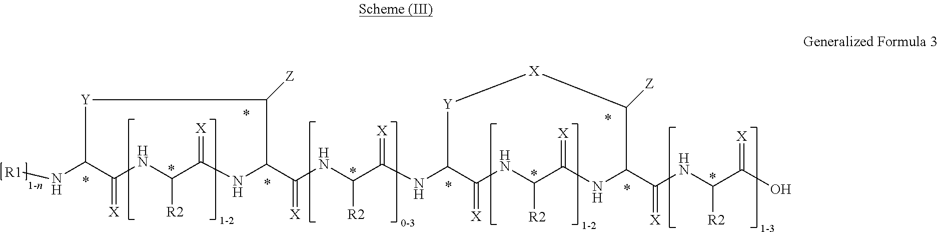

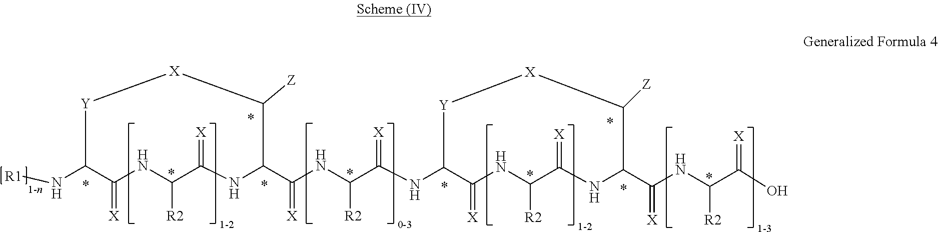

The above Schemes (I)-(IV) contain substituents and indexes corresponding to values R1, R2, X, Y, Z, and n. Herein, instances of R1 at any position of R1 are independent from any other position of R1, and can represent a hydrogen or an N-terminal extension by any number and combination of natural and/or non-natural amino acid(s), wherein n represents a variable number of amino acids as an extension; in various embodiments, n is 1, 2, 3, 4, 5, 6, 7, 8, 9, 10, or more than 10. Contained below are structures of 23 natural amino acids listed within Scheme, (Va-Vw). Within Scheme 6, 84 examples of non-natural amino acids are shown (VI1-VI84), this list is not meant to restrict the scope of the invention.

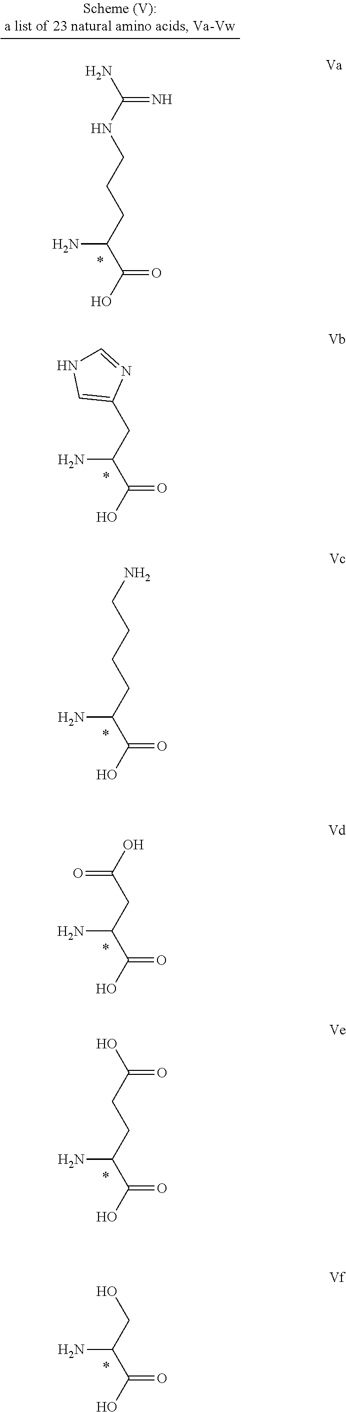 [SMILES: N=C(N)NCCCC(N)C(=O)O ; NC(Cc1c[nH]cn1)C(=O)O ; NCCCCC(N)C(=O)O ; NC(CC(=O)O)C(=O)O ; NC(CCC(=O)O)C(=O)O ; NC(CO)C(=O)O]

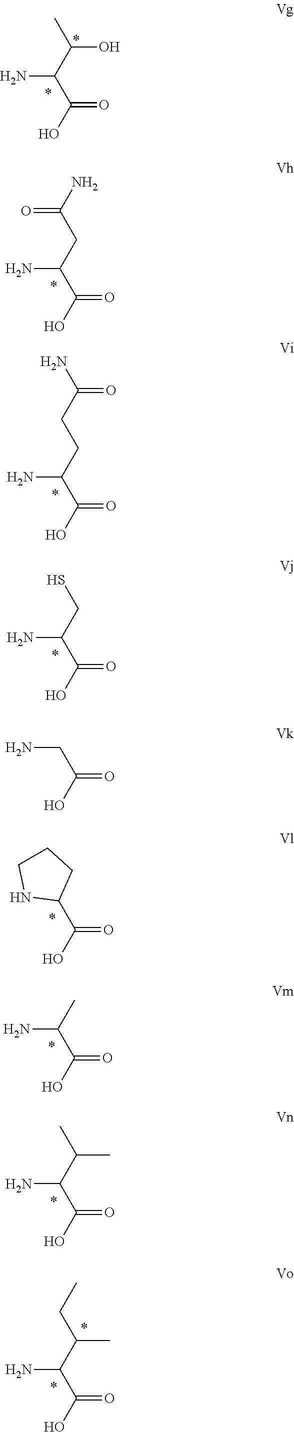

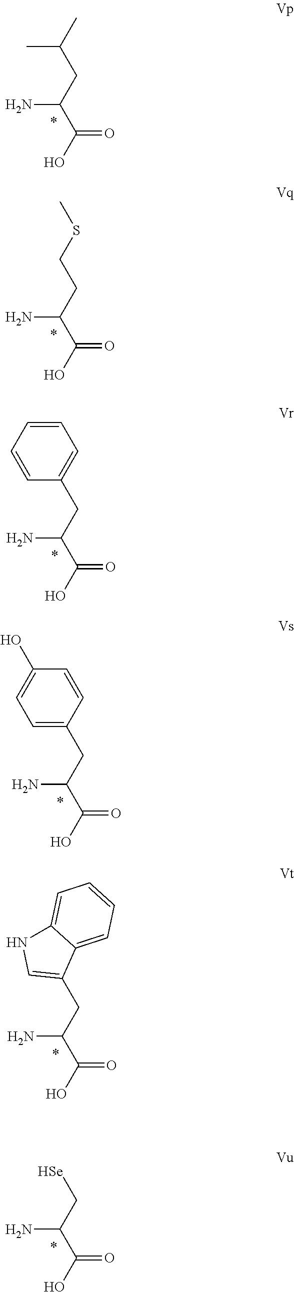

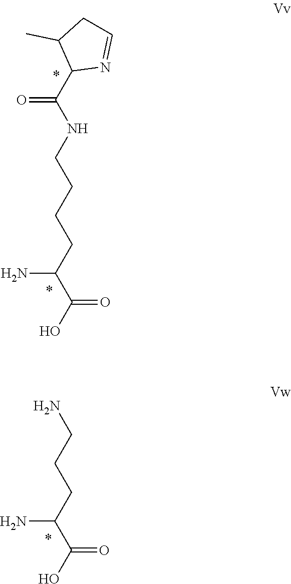 [SMILES: CC1CC=NC1C(=O)NCCCCC(N)C(=O)O ; NCCCC(N)C(=O)O]

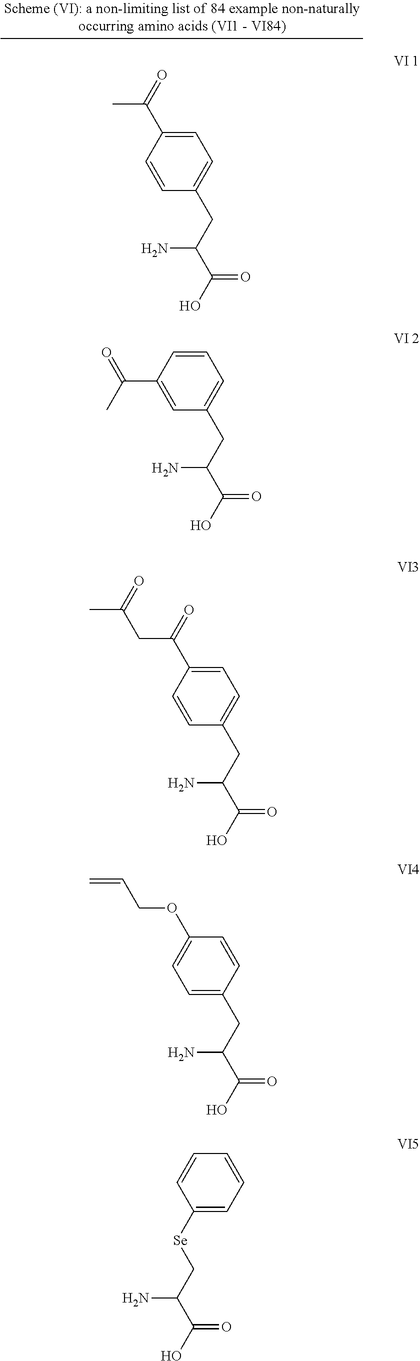

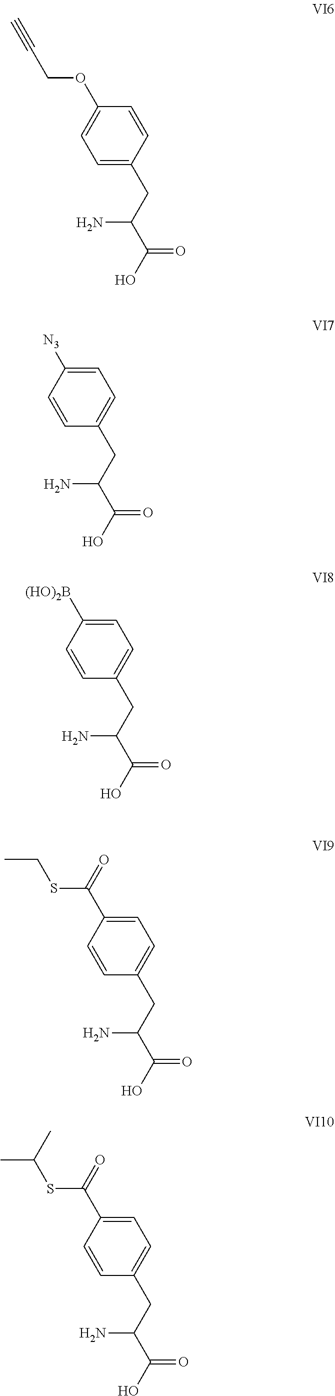 [SMILES: C#CCOc1ccc(CC(N)C(=O)O)cc1 ; [N-]=[N+]=Nc1ccc(CC(N)C(=O)O)cc1 ; NC(Cc1ccc(B(O)O)cc1)C(=O)O ; CCSC(=O)c1ccc(CC(N)C(=O)O)cc1 ; CC(C)SC(=O)c1ccc(CC(N)C(=O)O)cc1]

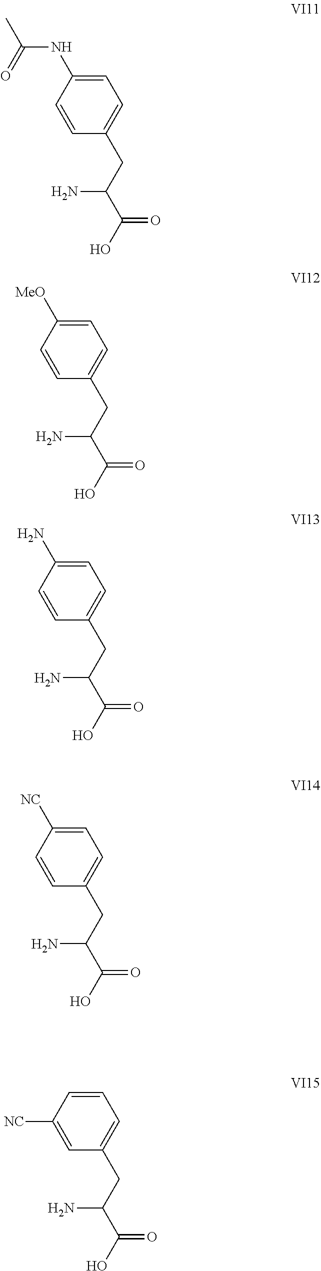 [SMILES: CC(=O)Nc1ccc(CC(N)C(=O)O)cc1 ; COc1ccc(CC(N)C(=O)O)cc1 ; Nc1ccc(CC(N)C(=O)O)cc1 ; N#Cc1ccc(CC(N)C(=O)O)cc1 ; N#Cc1cccc(CC(N)C(=O)O)c1]

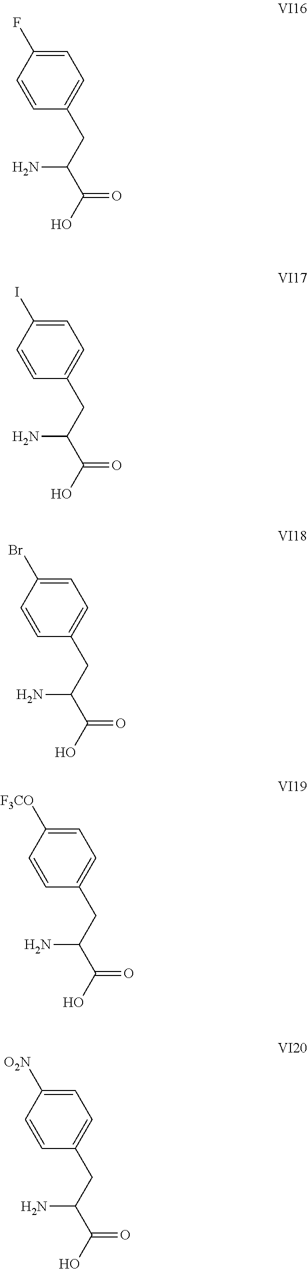 [SMILES: NC(Cc1ccc(F)cc1)C(=O)O ; NC(Cc1ccc(I)cc1)C(=O)O ; NC(Cc1ccc(Br)cc1)C(=O)O ; NC(Cc1ccc(OC(F)(F)F)cc1)C(=O)O ; NC(Cc1ccc([N+](=O)[O-])cc1)C(=O)O]

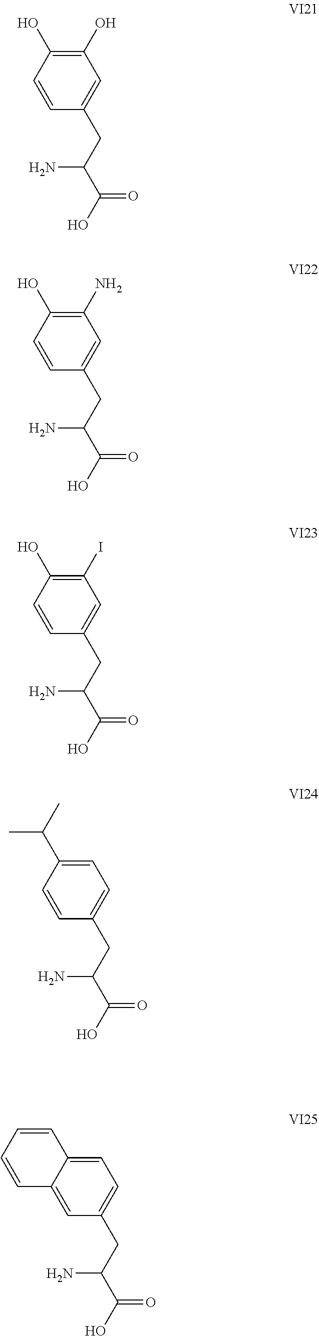 [SMILES: NC(Cc1ccc(O)c(O)c1)C(=O)O ; Nc1cc(CC(N)C(=O)O)ccc1O ; NC(Cc1ccc(O)c(I)c1)C(=O)O ; CC(C)c1ccc(CC(N)C(=O)O)cc1 ; NC(Cc1ccc2ccccc2c1)C(=O)O]

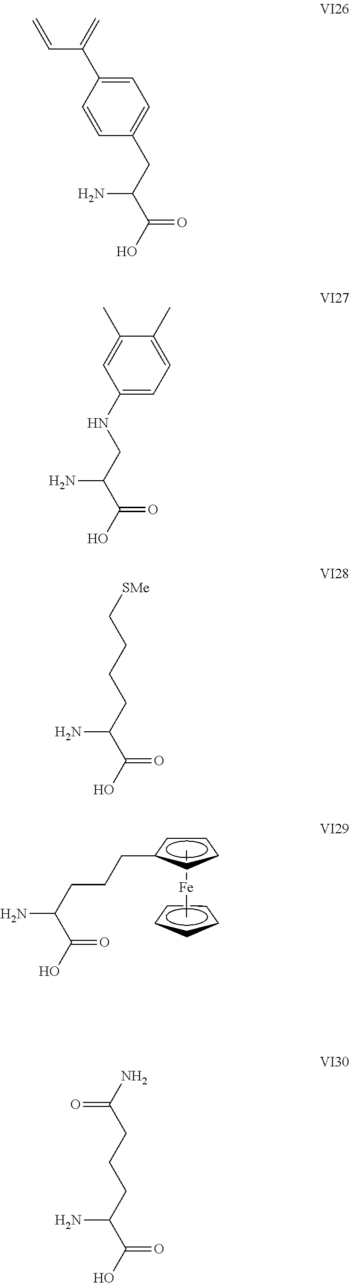

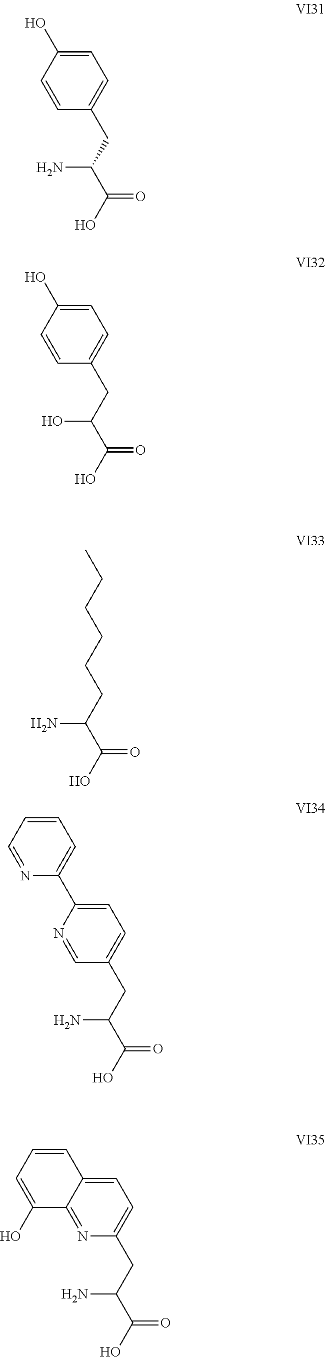

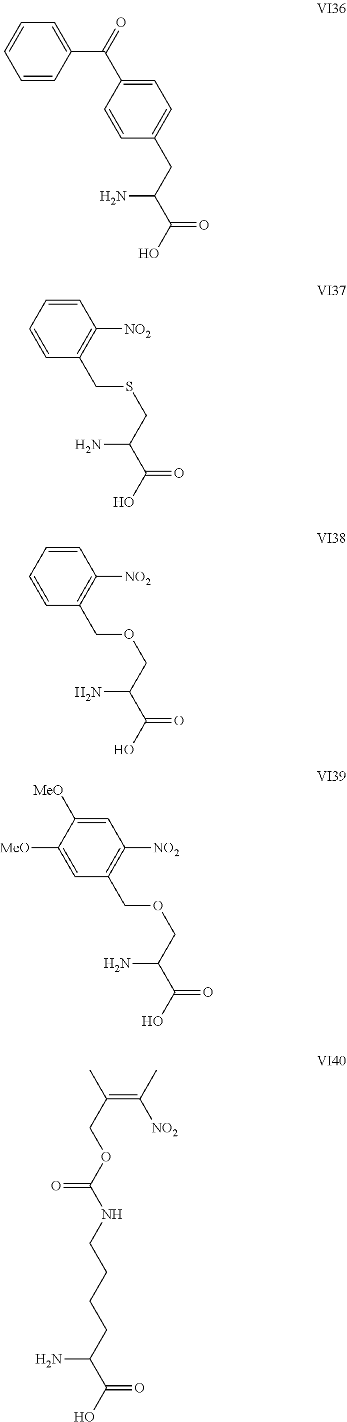 [SMILES: NC(Cc1ccc(C(=O)c2ccccc2)cc1)C(=O)O ; NC(CSCc1ccccc1[N+](=O)[O-])C(=O)O ; NC(COCc1ccccc1[N+](=O)[O-])C(=O)O ; COc1cc(COCC(N)C(=O)O)c([N+](=O)[O-])cc1OC ; C/C(COC(=O)NCCCCC(N)C(=O)O)=C(\C)[N+](=O)[O-]]

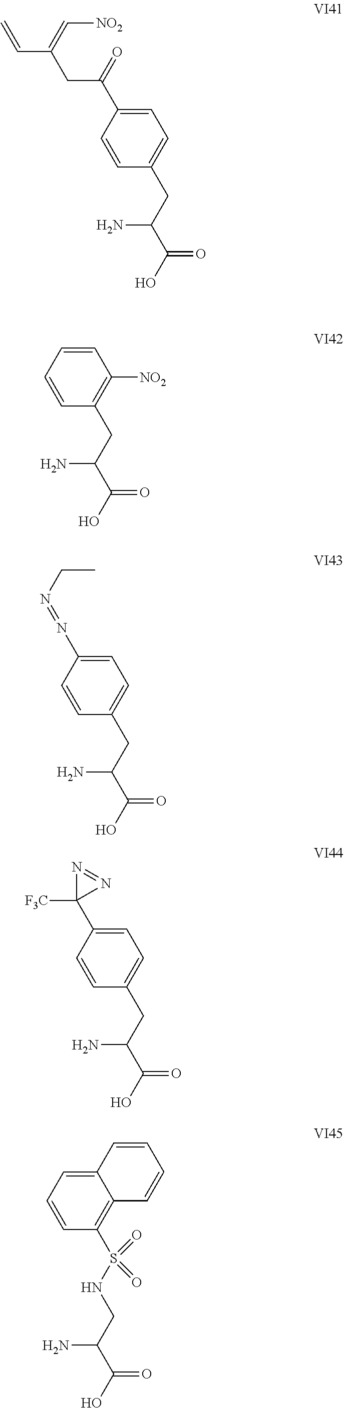

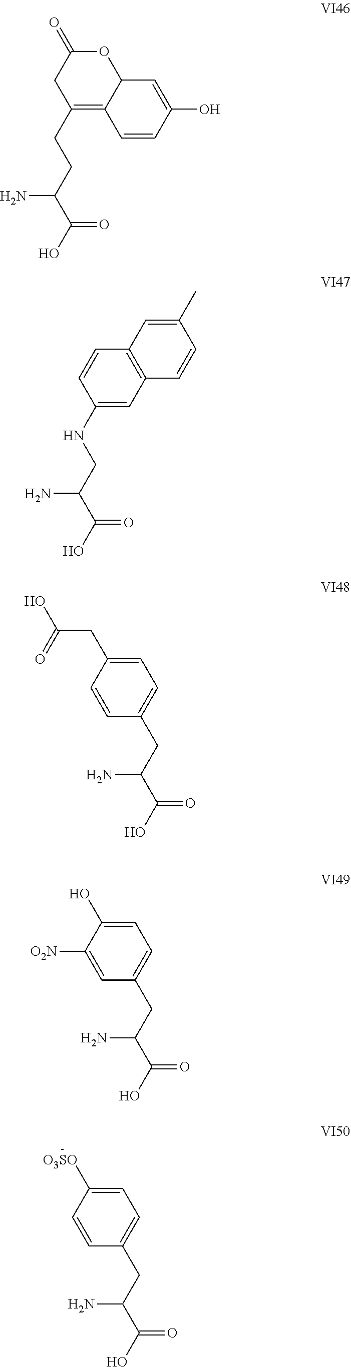

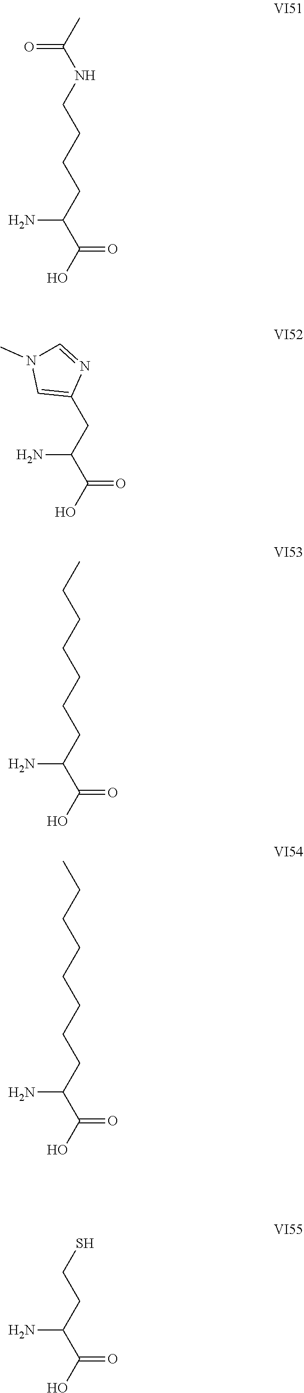

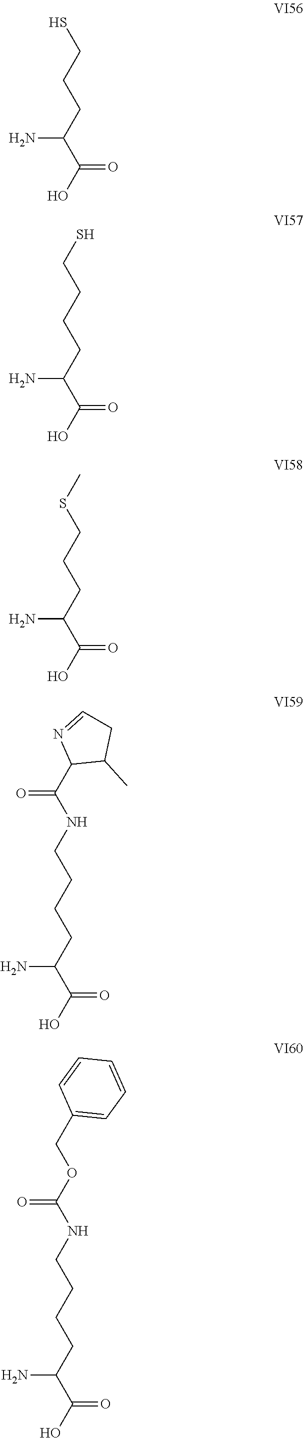

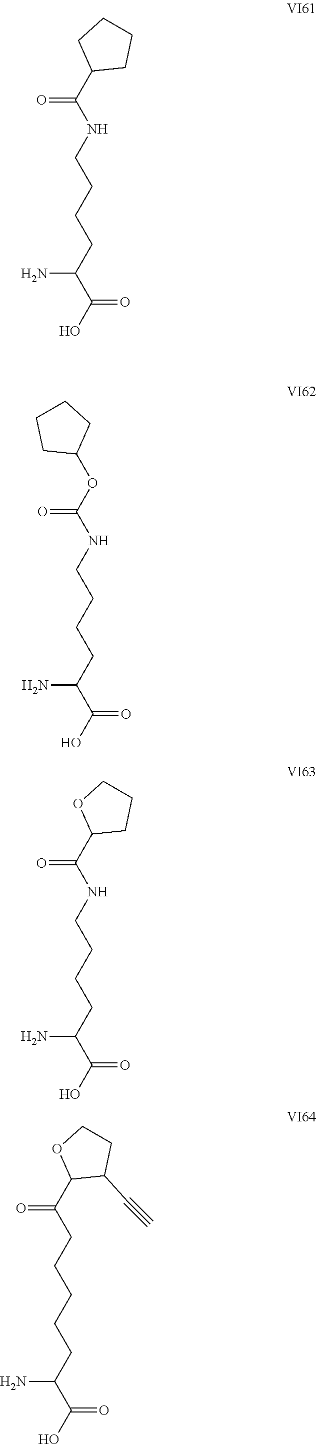

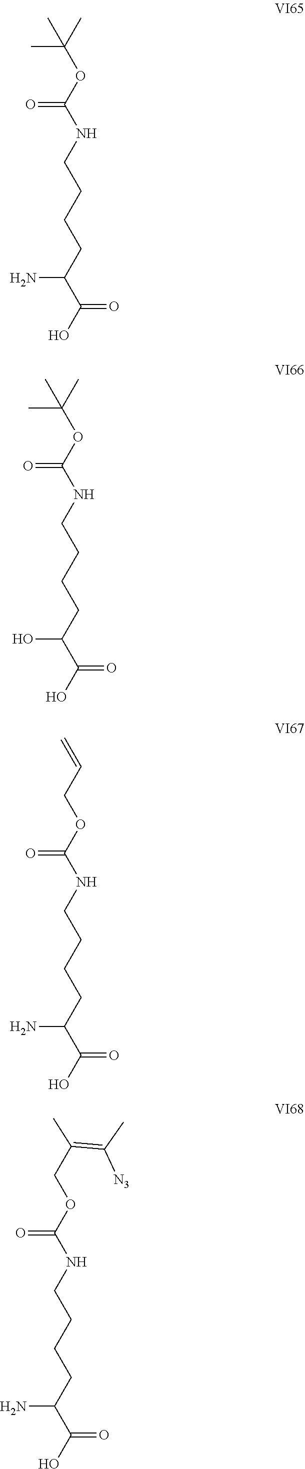

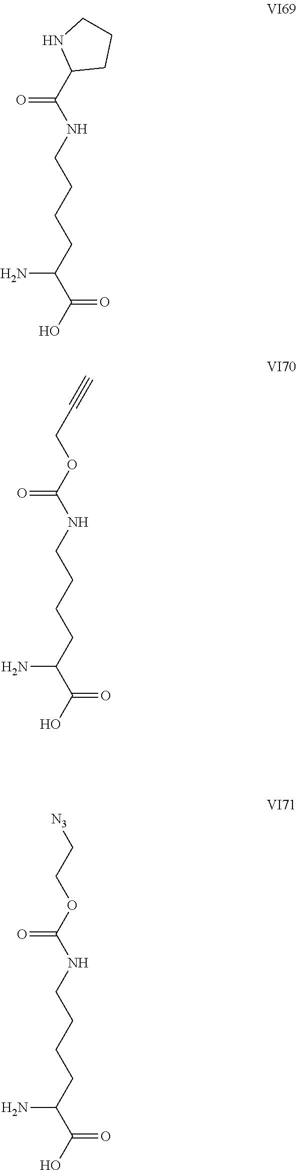 [SMILES: NC(CCCCNC(=O)C1CCCN1)C(=O)O ; C#CCOC(=O)NCCCCC(N)C(=O)O ; [N-]=[N+]=NCCOC(=O)NCCCCC(N)C(=O)O]

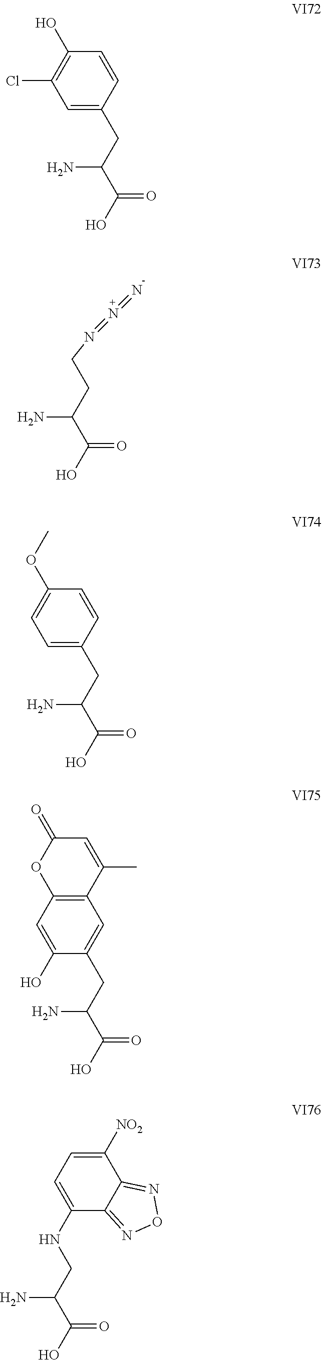

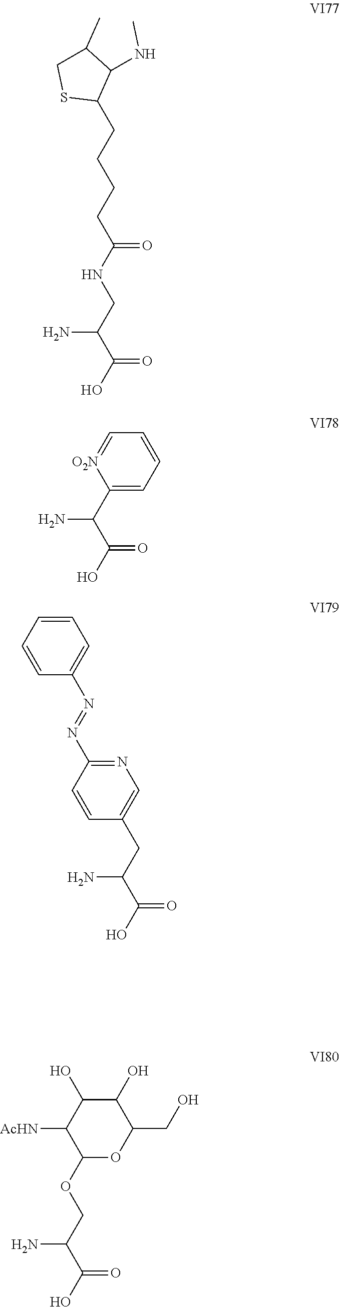

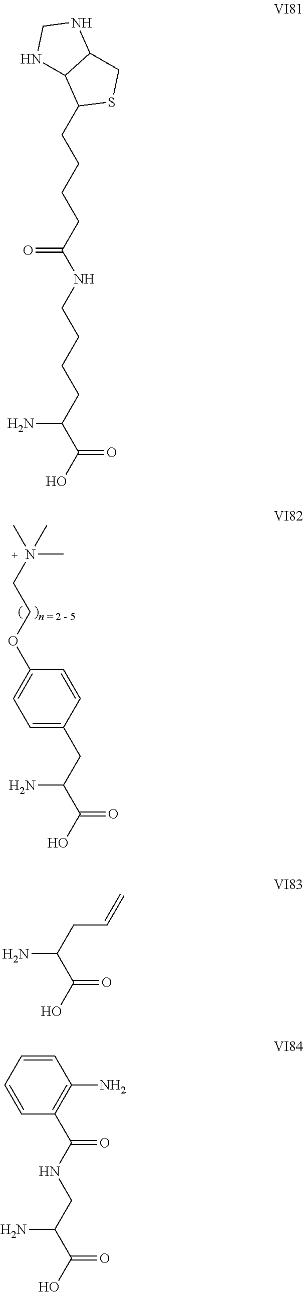

Pertaining to Scheme (I)-(IV), independent instances of R2 at any position of R2 are independent from any other position of R2, and R2 may indicate a side chain of a natural and/or non-natural amino acid. Herein, independent instances of X at any position of X are independent from any other position of X, and are used to represent either an oxygen (O) or sulfur (S); independent instances of Y at any position of Y are independent from any other position of Y, and represent any aromatic amino acid sidechain (Trp, His, Phe, Tyr), which participates in macrocyclization to a neighboring β-carbon moiety, further illustrated by the below substructures (Y.I-Y.VII); and independent instances of Z at any position of Z are independent from any other position of Z, and represent the sidechain of an amino acid beginning after the β-carbon, including natural and non-natural amino acid(s) that contain a β-carbon (i.e. excludes glycine).

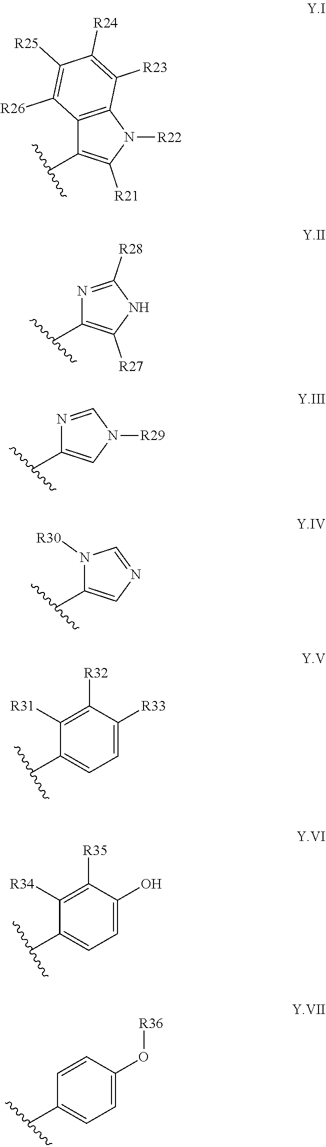

### Cyclophane Substructures of Bicyclic Peptides (Y.I-Y.VII)

The above substructures (Y.I-Y.VII) illustrate possible cyclization points for cyclophane formation, indicated by R21-R36, and embody aspects of Schemes (I-IV) at positions of Y, and R21 and R36 are each independently selected from the group consisting of hydrogen, alkyl, alkenyl, alkynyl, hydroxyl, hydroxyalkyl, halogen, —CN, —O-alkyl, —C(O)-alkyl, —C(O)O-alkyl, —C(O)OH, —C(O)NH₂, —C(O)NH-alkyl, —NH₂, —NO₂, —CF₃, —NH-alkyl, —N—(alkyl)₂, —NHC(O)-alkyl and aryl, wherein said alkyl, alkenyl, alkynyl and aryl are each optionally substituted.

Yet another aspect of the invention relates to a method for producing the compounds encoded by homologous biosynthetic clusters, or an enantiomer, diastereomer, tautomer, rotamer, racemates, prodrug, hydrate, or pharmaceutically acceptable salt thereof, the method comprising cultivating a bacterial isolate, one with such homologous cluster, in a culture comprising resources of carbon, nitrogen, and inorganic salts, thereby producing the compounds of the invention, corresponding to or analogous in structure to Formulae (I)-(XIII). In some embodiments, the method further includes the isolation of the compounds from the culture.

In yet another embodiment, the present invention relates to the compounds of the invention, or an enantiomer, diastereomer, tautomer, rotamer, racemates, prodrug, or pharmaceutically acceptable salt thereof, prepared according to the method described herein. In some embodiments, ¹³C, ²H, ¹⁸O, or ⁵N may be incorporated into the compounds of Formulae (I)-(XIII) by cultivation of bacterial isolates with carbon sources, nitrogen sources, water, and inorganic salts labeled with respective heavy element(s) under aerobic conditions, thereby producing or modifying compounds of Formulae (I)-(XIII) that contain the respective heavy element(s). In some embodiments, the method further includes the isolation of such compounds from the culture.

The present invention also relates to methods of preventing and/or treating disorders in a subject, e.g.; a human, in need thereof. The method includes the administration of a therapeutically effective amount of compounds described herein (e.g. FIG. 9), e.g., a compound of Formulae (I-XIII), to a subject, thereby treating the disorder in the subject. In some embodiments, the subject is a mammal (e.g. FIG. 9), a human, an animal, or a plant. In a specific embodiment, the subject is a human. In certain embodiments, the disorder is caused by an agent such as, but not limited to, a bacterium, a fungus, a virus, a protozoan, a helminth, a parasite, and combinations thereof.

In yet another embodiment, the present invention relates to compounds of one or more of Formula (I), (II), (III), (IV), (V), or (VI) and characterized by a monoisotopic mass of about (I) 1304.57, (II) 1391.60, (III) 1488.65, (IV) 1601.74, (V) 1702.79, or (VI) 1773.82 (FIGS. 5-8).

In general, as used herein, the term “substantially” means±10%, and in some embodiments, ±5%. In addition, reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

FIG. 1 graphically depicts the biosynthetic cluster of the RiPP Operon relevant to Formulae (I)-(XIII).

FIG. 2 illustrates closely related biosynthetic clusters in genomes of entomopathogenic bacteria in the form of a propeptide multisequence alignment.

FIG. 3 depicts non-limiting examples of the core region of related RiPPs embodied by the compounds of the invention.

FIG. 4 is a chromatogram illustrating RP-HPLC peaks and corresponding biological activity against Pseudomonas aeruginosa PA01 of compounds in accordance with Formulae (I)-(VI).

FIG. 5 is a high-resolution mass analysis of Formula (I).

FIG. 6 is a high-resolution mass analysis of Formula (III).

FIG. 7 is a high-resolution mass analysis of Formula (IV).

FIG. 8 is a high-resolution mass analysis of Formula (VI).

FIGS. 9 (A)-(C) shows treatment efficacy in an Escherichia coli AR350 septicemia mouse model. Mice which received doses compound of formula (I) received complete protection from multi-drug resistant E. coli strain AR350. Mice which received no intervention did not survive 24 hours. FIG. 9(A) shows time-dependent killing of E. coli 25922 in biological triplicate for the compound represented by Formula (I), darobactin, and ampicillin at 4×their respective MICs. Time points are graphed as the mean colony forming units (CFU)+/−the standard deviation. FIG. 9(B) shows the results in which mice were inoculated with a lethal dose of multi-drug resistant E. coli AR350, followed by administration of a single intraperitoneal dose of antibiotics at one hour post-infection. Four mice were tested per group. FIG. 9(C) shows the results in a neutropenic thigh model of E. coli AR350 infection, drug s were delivered to mice (n=4) by intraperitoneal injection two hours post-infection. At 24 hours, thighs were homogenized, serially diluted, and plated in triplicate for CFU.

FIG. 10 shows the 3D cryoEM microED-generated structure of compound of Formula (I).

FIG. 11 is a table of data collection and refinement statistics for cryoEM microED.

FIG. 12 is a molecular networking chart [M+H]⁺ corresponding to compounds of Formulae (I)-(VI), which are embodied by Scheme (I), and others embodied by Scheme (II-IV).

FIG. 13A is a proton NMR spectrum of the compound represented by Formula (I). FIG. 13B is a carbon NMR spectrum of the compound represented by Formula (I).

FIG. 14A is a 2D NMR spectra recorded in D₂0 of the compound represented by Formula (I). FIG. 14B is a Key 2D NMR correlations in D₂0 and DMSO-d6.

FIGS. 15A-15C are solution NMR spectroscopy of BamA-β interacting with the compound represented by Formula (I). FIG. 15A is a 2D [¹⁵N, ¹H]-TROSY spectra of apo BamA-β in LDAO micelles overlaid with BamA-β with 1.0 eq of the compound represented by Formula (I). Zoomed-in panels show selected resonances. Tentatively assigned W810 is indicated with a frame on the spectrum. FIG. 15B is a 2D [¹⁵N, ¹H]-TROSY spectra of BamA-β in a titration experiment with increasing concentration of the compound represented by Formula (I). FIG. 15C is a NMR spectrum of mutant W810F to confirm the assignment of W810.

FIG. 16 is a Marfey's analysis of the compound represented by Formula (I).

### DETAILED DESCRIPTION

The present invention relates generally to the novel ribosomally-produced post-translationally modified class of molecules, compounds of the invention (Dynobactins), to the processes for preparation of the compounds of the invention, to pharmaceutical compositions comprising these compounds of the invention, and to methods of using the compounds of the invention to treat, ameliorate, and/or prevent various disorders, e.g., bacterial infections. The present invention relates to a class of novel antibiotics which have activity against numerous Gram-negative pathogens, including strains resistant to antibiotics. The compounds of the invention disclosed herein also have favorable bioavailability and low toxicity.

As used herein and unless otherwise indicated, the term “compounds of the invention” means, collectively, the compounds of Formulae (I)-(XIII), generalized formulae embodied by Schemes (I)-(IV), and pharmaceutically acceptable salts thereof as well as specific compounds depicted herein. The compounds of the invention are identified herein by their chemical structure and/or chemical name. Where a compound is referred to by both a chemical structure and a chemical name, and that chemical structure and chemical name conflict, the chemical structure is determinative of the compound's identity. The compounds of the invention may contain one or more chiral centers and/or double bonds and, therefore, exist as stereoisomers, such as double-bond isomers (i.e., geometric isomers), enantiomers, or diastereomers. According to the invention, the chemical structures depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically- or enantiomerically-pure intermediates, reagents, and catalysts by well-known asymmetric synthetic methods. The compounds of the invention are effective against important Gram-negative pathogens. These compounds have good activity against Escherichia coli, Pseudomonas aeruginosa, Klebsiella pneumoniae, Salmonella Typhimurium and Shigella sonnei. Moreover, the compounds of the invention lack activity against Gram-positive pathogens and are inactive against Gram-negative intestinal symbionts, Bacteroides. As detailed below, this selectivity is pharmacologically beneficial. Based on the unusual structure of dynobactin A, including a C—C link between tryptophan and asparagine and N—C link between histidine and tyrosine, compounds of the invention belong to a novel class of antimicrobial agents. Indeed, the last new class of compounds acting against Gram-negative bacteria to reach hospitals was discovered 50 years ago.

The ribosomal encoding enables production of analogs of any of Formulae (I)-(XIII) by nucleotide substitution in the gene coding for the precursor peptide. Such substitution may be achieved using any of various standard biochemical methods. Synthesis of the oligonucleotides with both specific and random substitutions of nucleotides of the coding region will produce a large array of fragments. These oligonucleotides will be ligated with upstream and downstream sequences coding for the precursor peptide, cloned into an expression vector, and transformed into cells carrying the operon with a disrupted precursor peptide gene. Analogs may be isolated from clones of this recombinant library and tested for activity. Analogs with increased potency against bacteria or improved pharmacological properties may be isolated and developed into drugs. This substitution approach is possible at the positions indicated in Schemes (I)-(IV) for natural amino acids specified within Scheme (V) or non-natural amino acids specified within Scheme (VI). Incorporation of non-natural amino acids is made possible through stop codon substitution and tRNA evolution techniques established within the field of protein engineering. Selective activity against Gram-negative pathogens is highly unusual, and in fact, there is only one clinically-used antibiotic class with such properties—the polymyxins, which act by binding to the bacterial lipopolysaccharides (LPS). Compounds in accordance herewith do not bind to LPS and, importantly, are active against polymyxin-resistant mutants. Polymyxin is the antibiotic of last resort against multidrug resistant (MDR) Gram-negative bacteria.

As used herein and unless otherwise indicated, the term “alkyl” means a substituted or unsubstituted, saturated, linear or branched hydrocarbon chain radical. Examples of alkyl groups include, but are not limited to, C₁₋₁₅ linear, branched or cyclic alkyl, such as methyl, ethyl, propyl, isopropyl, cyclopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, sec-butyl, t-butyl, cyclobutyl, pentyl, isopentyl, neopentyl, hexyl, and cyclohexyl and longer alkyl groups, such as heptyl, octyl, nonyl and decyl. An alkyl can be unsubstituted or substituted with one or two suitable substituents.

As used herein and unless otherwise indicated, the terms “alkoxy” or “alkyloxy” means an —O-alkyl, wherein alkyl is as defined herein. An alkoxy can be unsubstituted or substituted with one or two suitable substituents. Preferably, the alkyl chain of an alkyloxy is from 1 to 5 carbon atoms in length, referred to herein, for example, as “C₁₋₅ alkoxy.” In one embodiment, the alkyl chain of an alkyloxy is from 1 to 10 carbon atoms in length, referred to herein, for example, as “C₁₋₁₀ alkoxy.”

As used herein and unless otherwise indicated, the terms “alkene” or “alkenyl group” means a monovalent linear, branched or cyclic hydrocarbon chain having one or more double bonds therein. The double bond of an alkene can be unconjugated or conjugated to another unsaturated group. An alkene can be unsubstituted or substituted with one or two suitable substituents. Suitable alkenes include, but are not limited to C₂₋₈ alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkene can be unsubstituted or substituted with one or two suitable substituents.

As used herein and unless otherwise indicated, the terms “alkynyl” means an unsaturated straight or branched hydrocarbon having at least one carbon-carbon triple bond. Examples of alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

As used herein and unless otherwise indicated, the term “aryl” or “aromatic ring” means a monocyclic or polycyclic conjugated ring structure that is well known in the art. Examples of suitable aryl groups or aromatic rings include, but are not limited to, phenyl, tolyl, anthacenyl, fluorenyl, indenyl, azulenyl, and naphthyl. An aryl group can be unsubstituted or substituted with one or two suitable substituents. In one embodiment, the aryl group is a monocyclic ring, wherein the ring comprises 6 carbon atoms, referred to herein as “C₆ aryl.”

“Substituted aryl” includes an aryl group optionally substituted with one or more functional groups, such as halo, alkyl, haloalkyl (e.g., trifluoromethyl), alkoxy, haloalkoxy (e.g., difluoromethoxy), alkenyl, alkynyl, aryl, heteroaryl, arylalkyl, aryloxy, aryloxyalkyl, arylalkoxy, alkoxycarbonyl, alkylcarbonyl, arylcarbonyl, arylalkenyl, aminocarbonylaryl, arylthio, arylsulfinyl, arylazo, heteroarylalkyl, heteroarylalkenyl, heteroaryloxy, hydroxy, nitro, cyano, amino, substituted amino wherein the amino includes 1 or 2 substituents (which are optionally substituted alkyl, aryl or any of the other substituents recited herein), thiol, alkylthio, arylthio, heteroarylthio, arylthioalkyl, alkoxyarylthio, alkylaminocarbonyl, arylaminocarbonyl, aminocarbonyl, alkylcarbonyloxy, arylcarbonyloxy, alkylcarbonylamino, arylcarbonylamino, arylsulfinyl, arylsulfinylalkyl, arylsulfonylamino, or arylsulfonaminocarbonyl and/or any of the alkyl substituents recited herein.

The term “heteroaryl” as used herein alone or as part of another group refers to a 5- to 7-membered aromatic ring which includes 1, 2, 3 or 4 hetero atoms such as nitrogen, oxygen or sulfur and such rings fused to an aryl, cycloalkyl, heteroaryl or heterocycloalkyl ring (e.g. benzothiophenyl, indolyl), and includes possible N-oxides. “Substituted heteroaryl” includes a heteroaryl group optionally substituted with 1 to 4 substituents, such as the substituents included above in the definition of “substituted alkyl” and “substituted cycloalkyl.” Substituted heteroaryl also includes fused heteroaryl groups which include, for example, quinoline, isoquinoline, indole, isoindole, carbazole, acridine, benzimidazole, benzofuran, isobenzofuran, benzothiophene, phenanthroline, purine, and the like.

Moreover, the terms “heterocyclo,” “heterocycle,” or “heterocyclic ring,” as used herein, refer to an unsubstituted or substituted stable 5- to 7-membered monocyclic ring system which may be saturated or unsaturated, and which consists of carbon atoms and from one to four heteroatoms selected from N, O or S, and wherein the nitrogen and sulfur heteroatoms may optionally be oxidized, and the nitrogen heteroatom may optionally be quaternized. The heterocyclic ring may be attached at any heteroatom or carbon atom which results in the creation of a stable structure. Examples of such heterocyclic groups include, but are not limited to, piperidinyl, piperazinyl, oxopiperazinyl, oxopiperidinyl, oxopyrrolidinyl, oxoazepinyl, azepinyl, pyrrolyl, pyrrolidinyl, furanyl, thienyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isooxazolyl, isoxazolidinyl, morpholinyl, thiazolyl, thiazolidinyl, isothiazolyl, thiadiazolyl, tetrahydropyranyl, thiamorpholinyl, thiamorpholinylsulfoxide, thiamorpholinylsulfone, and oxadiazolyl.

The term “cycloalkyl” includes saturated or partially unsaturated (containing 1 or more double bonds) cyclic hydrocarbon groups containing 1 to 3 rings, including monocyclicalkyl, bicyclicalkyl and tricyclicalkyl, containing a total of 3 to 20 carbons forming the rings, or about 3 to 10 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl, cyclododecyl, and cyclohexenyl.

“Substituted cycloalkyl” includes a cycloalkyl group optionally substituted with 1 or more substituents such as halogen, alkyl, substituted alkyl, alkoxy, hydroxy, aryl, substituted aryl, aryloxy, cycloalkyl, alkylamido, alkanoylamino, oxo, acyl, arylcarbonylamino, amino, nitro, cyano, thiol and/or alkylthio and/or any of the substituents included in the definition of “substituted alkyl.”

The term “cycloalkenyl” includes a nonaromatic monocyclic or bicyclic carbocylic ring containing at least one double bond. Examples of cycloalkenyl groups include, but are not limited to, cyclopropenyl, cyclobutenyl, cyclopentenyl, cyclohexenyl, cycloheptenyl, cyclooxtenyl and the like.

As used herein and unless otherwise indicated, the term “aryloxy” means an —O-aryl group, wherein aryl is as defined herein. An aryloxy group can be unsubstituted or substituted with one or two suitable substituents. Preferably, the aryl ring of an aryloxy group is a monocyclic ring, wherein the ring comprises 6 carbon atoms, referred to herein as “_{C} aryloxy.”

As used herein and unless otherwise indicated, the term “ether” means a group of formula alkyl-O-alkyl, alkyl-O-alkynyl, alkyl-O-aryl, alkenyl-O-alkenyl, alkenyl-O-alkynyl, alkenyl-O-aryl, alkynyl-O-alkynyl, alkynyl-O-aryl, aryl-O-aryl, wherein “alkyl”, “alkenyl”, “alkynyl” and “aryl” are defined herein.

As used herein and unless otherwise indicated, the term “carboxy” means a radical of the formula: —COOH.

As used herein and unless otherwise indicated, the term “halogen” means fluorine, chlorine, bromine, or iodine. Correspondingly, the meaning of the terms “halo” and “Hal” encompass fluoro, chloro, bromo, and iodo.

As used herein and unless otherwise indicated, the terms “substituted,” “optionally substituted” and “suitable substituent” mean groups that do not nullify the synthetic or pharmaceutical utility of the compounds of the invention or the intermediates useful for preparing them. Examples of substituted groups or suitable substituents include, but are not limited to: C₁₋₁₀ alkyl; C₁₋₁₀ alkenyl; C₁₋₁₀ alkynyl; C₆ aryl; C₃₋₅ heteroaryl; C₃₋₇ cycloalkyl; C₁₋₁₀ alkoxy; C₆ aryloxy; —CN; —OH; SH; oxo; halo; —NO₂; —CO₂H; —NH₂; —NHOH; —NH(C₁₋₁₀ alkyl); —N(C₁₋₁₀ alkyl)₂; —NH(C₆ aryl); —NHO(C₁₋₁₀ alkyl); —N(OC₁₋₁₀ alkyl)₂; —NH(OC₆ aryl); —S(C₁₋₁₀ alkyl); —S(C₆ aryl); (═O); —N(C₆ aryl)₂; —CHO; —C(O)(C₁₋₁₀ alkyl); —C(O)(C₆ aryl); —C(O)O(C₁₋₁₀ alkyl); and —C(O)O(C₆ aryl), —C(S)(C₁₋₁₀ alkyl); —C(S)(C₆ aryl); —SO₂(C₁₋₁₀ alkyl); —SO₂(C₆ aryl), —SO(C₁₋₁₀ alkyl); —SO(C₆ aryl), and —SO₃H, —C(S)O(C₁₋₁₀ alkyl); —C(S)OC₆ aryl.

In certain illustrative embodiments, the substituents can be one or more than one suitable groups, such as, but not limited to, —F, —Cl, —Br, —I, —OH, azido, —SH, alkyl, aryl, heteroalky, alkyoxyl, alkylthiol, amino, hydroxylamino, N-alkylamino, —N,N-dialkylamino, —N,N-dimethylamino, acyl, alkyloxycarbonyl, sulfonyl, urea, —NO₂, and triazolyl. One of skill in art can readily choose a suitable substituent based on the stability and pharmacological and synthetic activity of the compound of the invention.

The phrase “pharmaceutically acceptable salt(s),” as used herein includes but is not limited to salts of acidic or basic groups that may be present in the compounds (including the compounds of the invention) used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts and, particularly, calcium, magnesium, sodium, lithium, zinc, potassium and iron salts.

As used herein, the term “prodrug” or “pharmaceutically acceptable prodrug” means a derivative of a compound that can hydrolyze, oxidize, or otherwise react under biological conditions, in vitro or in vivo, to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include but are not limited to compounds that comprise oligonucleotides, peptides, lipids, aliphatic and aromatic groups, or NO, NO₂, ONO, and ONO₂ moieties. Prodrugs can typically be prepared using well known methods.

As used herein, the term “hydrate” means a compound or a salt thereof that further includes a stoichiometric or non-stoichiometric amount of water bound by non-covalent intermolecular force.

In one aspect, provided herein are methods of treating, ameliorating, or preventing a bacterial infection in a subject in need thereof, comprising administering to the subject a therapeutically effective amount of at least one of the compounds of Formula (I), Formula (Ia) or Formula (II), or at least one of the specific compounds described herein. Administration of the compound may be topical, such as subcutaneous, transdermal, rectal, intravaginal, intranasal, intrabronchial, intraocular, or intra-aural. Alternatively, administration may be systemic, such as oral administration. In still other alternatives, administration may be parenteral, intravenous, intramuscular, or intraperitoneal.

As used herein, the term “administration” can also include administering a combination of compounds. Thus, administration may be in the form of dosing an organism with a compound or combination of compounds, such that the organism's circulatory system will deliver a compound or combination of compounds to the target area, including but not limited to a cell or cells, synaptic junctions and circulation. Administration may also mean that a compound or combination of compounds is placed in direct contact with an organ, tissue, area, region, cell or group of cells, such as but not limited to direct injection of the combination of compounds.

In select embodiments, a combination of compounds can be administered, and thus the individual compounds can also be said to be co-administered with one another. As used herein, “co-administer” indicates that each of at least two compounds is administered during a time frame wherein the respective periods of biological activity or effects overlap. Thus the term co-administer includes sequential as well as coextensive administration of the individual compounds, at least one of which is a compound of the present invention. Accordingly, “administering” a combination of compounds according to some of the methods of the present invention includes sequential as well as coextensive administration of the individual compounds of the present invention. Likewise, the phrase “combination of compounds” indicates that the individual compounds are co-administered, and the phrase “combination of compounds” does not mean that the compounds must necessarily be administered contemporaneously or coextensively. In addition, the routes of administration of the individual compounds need not be the same.

As used herein, the terms “treat” and “treatment” refer to a slowing of or a reversal of the progress of the disease or infection. Treating a disease includes treating a symptom and/or reducing the symptoms of the disease or infection. The term “preventing” refers to a slowing of the disease or of the onset of the disease, infection or the symptoms thereof. Preventing a disease or infection can include stopping the onset of the disease, infection or symptom thereof.

As used herein, the term “subject” may be an animal, vertebrate animal, mammal, rodent (e.g., a guinea pig, a hamster, a rat, a mouse), a murine (e.g., a mouse), a canine (e.g., a dog), a feline (e.g. a cat), an equine (e.g., a horse), a primate, a simian (e.g., a monkey or ape), a monkey (e.g., marmoset, a baboon), an ape (e.g., gorilla, chimpanzee, orangutan, gibbon), or a human.

As used herein, the term “pest” includes, but not limited to insects, fungi, bacteria, nematodes, mites, ticks and the like.

As used herein, the term “dosage unit” refers to a physically discrete unit, such as a capsule or tablet suitable as a unitary dosage for a subject. Each unit contains a predetermined quantity of a compound of the invention which was discovered or believed to produce the desired pharmacokinetic profile which yields the desired therapeutic effect. The dosage unit is composed of a compound of one or more of Formulae (I)-(XIII) and/or of Schemes (I)-(IV) the invention in association with at least one pharmaceutically acceptable carrier, salt, excipient or a combination thereof. The term “dose” or “dosage” refers to the amount of active ingredient that an individual takes or is administered at one time.

The term “therapeutically effective amount” refers to the amount sufficient to produce a desired biological effect in a subject. Accordingly, a therapeutically effective amount of a compound may be an amount which is sufficient to treat or prevent a disease or infection, and/or delay the onset or progression of a disease or infection, and or alleviate one or more symptoms of the disease or infection, when administered to a subject suffered from or susceptible to that disease or infection. The term “pesticidally effective amount” refers to the amount of pesticide able to bring about death to at least one pest, or noticeably reduce pest growth, feeding, or normal physiological development. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective polypeptide composition. A “pharmaceutically acceptable carrier” or “pharmaceutically acceptable excipient” herein refers to a non-API (where API refers to Active Pharmaceutical Ingredient) substances such as disintegrators, binders, fillers, and lubricants used in formulating pharmaceutical products. They are generally safe for administering to humans. An “agriculturally acceptable carrier” herein refers to all adjuvants, inert components, dispersants, surfactants, tackifiers, binders etc. that are ordinarily used in pesticide formulation technology; these are well known to those skilled in pesticide formulation.

The term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. In one embodiment, when administered to a patient, the combination of compounds of the invention and pharmaceutically acceptable vehicles are sterile. Water and/or oils are one vehicle when the combination of compounds of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present combination of compounds, if desired, can also contain minor amounts of wetting or emulsifying agents, or pH buffering agents.

In general, each of the individual compounds of the invention may also be administered by any convenient route, for example, orally, by infusion or bolus injection, or by absorption through epithelial or mucocutaneous linings (e.g., oral mucosa, rectal and intestinal mucosa, etc.), and may be administered together with another biologically active agent. Administration can be systemic or local. Various delivery systems are known, e.g., encapsulation in liposomes, microparticles, microcapsules, capsules, etc., and can be used to administer at least one of the compounds of the invention. Methods of administration of the individual compounds include but are not limited to intradermal, intramuscular, intraperitoneal, intravenous, subcutaneous, intranasal, epidural, oral, sublingual, intranasal, intracerebral, intravaginal, transdermal, rectal, pulmonary or topical, particularly to the ears, nose, eyes, or skin. The preferred mode of administration is left to the discretion of the practitioner, and will depend, in part, upon the site of the medical condition.

In specific embodiments, it may be desirable to administer one or more compounds of the combination locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327).

In yet another embodiment, at least one of the compounds used in the methods of the invention can be delivered in a controlled-release system. In one embodiment, a pump may be used (see Langer, supra; Sefton, 1987, CRC Crit. Ref Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of an organ, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Each of the individual compounds to be administered can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in Remington's Science and Practice of Pharmacy (21st ed., Hendrickson, R., et al., Eds., Lippincott Williams & Wilkins, Baltimore, MD (2006)), which is incorporated by reference.

Typically, when the individual compounds of the invention are administered intravenously, the compounds are in sterile isotonic aqueous buffered solutions. Where necessary, the individual compounds of the invention may also include a solubilizing agent. The individual compounds of the invention for intravenous administration may optionally include a local anesthetic such as lidocaine to ease pain at the site of the injection.

In one embodiment, individual compounds are supplied either together in a unit dosage form or separately. Regardless, compounds may be supplied, for example, as a dry lyophilized powder or water free concentrate in a hermetically sealed container such as an ampoule indicating the quantity of active agent. Where the compound or combination of compounds of the invention are to be administered by infusion, they can be dispensed, for example, with an infusion bottle containing sterile pharmaceutical grade water or saline. Where the compound or combination of compounds of the invention is administered by injection, an ampoule of sterile water for injection or saline can be provided so that the ingredients may be mixed prior to administration.

Compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Immediate release formulations for oral use include tablets or capsules containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, mannitol, sorbitol, acacia, alginic acid, sodium alginate, gelatin, starch, pregelatmized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like as are found, for example, in The Handbook of Pharmaceutical Excipients, third edition, edited by Arthur H. Kibbe, American Pharmaceutical Association Washington DC.

Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

For oral delivery, the active compounds can be incorporated into a formulation that includes pharmaceutically acceptable carriers such as binders (e.g., gelatin, cellulose, gum tragacanth), excipients (e.g., starch, lactose), disintegrating agents (e.g., alginate, Primogel, and corn starch), and sweetening or flavoring agents (e.g., glucose, sucrose, saccharin, methyl salicylate, and peppermint). The formulation can be orally delivered in the form of enclosed gelatin capsules or compressed tablets. The capsules and tablets can also be coated with various coating known in the art to modify the flavors, tastes, colors, and shapes of the capsules and tablets. The carrier may be solid or a liquid, or both, and may be formulated with at least one compound described herein as the active compound which may contain from about 0.05% to about 95% by weight of the at least one active compound. Suitable oral formulations can also be in the form of suspension, syrup, chewing gum, wafer, elixir, and the like.

If desired, conventional agents for modifying flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered parenterally in the form of solution or suspension, or in lyophilized form capable of conversion into a solution or suspension form before use. In such formulations, diluents or pharmaceutically acceptable carriers such as sterile water and physiological saline buffer can be used. Other conventional solvents, pH buffers, stabilizers, anti-bacteria agents, surfactants, and antioxidants can all be included. For example, useful components include sodium chloride, acetates, citrates or phosphates buffers, glycerin, dextrose, fixed oils, methyl parabens, polyethylene glycol, propylene glycol, sodium bisulfate, benzyl alcohol, ascorbic acid, and the like. The parenteral formulations can be stored in any conventional containers such as vials and ampoules.

Routes of topical administration include nasal, bucal, mucosal, rectal, or vaginal applications. For topical administration, the active compounds can be formulated into lotions, creams, ointments, powders, pastes, sprays, suspensions, drops and aerosols. Thus, one or more thickening agents, humectants, and stabilizing agents can be included in the formulations. Examples of such agents include, but are not limited to, polyethylene glycol, sorbitol, xanthan gum, petrolatum, beeswax, or mineral oil, lanolin, squalene, and the like. A special form of topical administration is delivery by a transdermal patch. Methods for preparing transdermal patches are disclosed, e.g., in Brown, et al. (1988) Ann. Rev. Med. 39:221-229 which is incorporated herein by reference. Carriers and excipients which may be used include Vaseline, lanoline, polyethylene glycol, alcohols, and combination of two or more thereof. The active compound is generally present at a concentration of from about 0.1% to about 80% w/w of the composition, for example from about 0.2% to 50%.

Subcutaneous implantation for sustained release of the active compounds may also be a suitable route of administration. This entails surgical procedures for implanting an active compound in any suitable formulation into a subcutaneous space, e.g., beneath the anterior abdominal wall. See, e.g., Wilson et al. (1984) J. Clin. Psych. 45:242-247. Hydrogels can be used as a carrier for the sustained release of the active compounds. Hydrogels are generally known in the art. They are typically made by crosslinking high molecular weight biocompatible polymers into a network, which swells in water to form a gel like material. Preferably, hydrogels are biodegradable or biosorbable. For purposes of this invention, hydrogels made of polyethylene glycols, collagen, or poly(glycolic-co-L-lactic acid) may be useful. See, e.g., Phillips et al. (1984) J Pharmaceut. Sci., 73: 1718-1720.

The amount of each individual compounds to be administered will depend on the nature or severity of the symptoms, and can be determined by standard clinical techniques. In addition, in vitro or in vivo assays may optionally be employed to help identify optimal dosage ranges for each of the components of the combination. The precise dose of each component to be employed will also depend on the route of administration and the seriousness of the disease or disorder, and a practitioner can determine these doses based upon each patient's circumstances. In general, however, suitable dosage ranges for oral administration are generally about 0.001 mg to 1000 mg of a compound of the invention per kilogram body weight. In specific embodiments of the invention, the oral dose for each component is 0.01 mg to 100 mg per kilogram body weight, more specifically 0.1 mg to 50 mg per kilogram body weight, more specifically 0.5 mg to 20 mg per kilogram body weight, and yet even more specifically 1 mg to 10 mg per kilogram body weight. The dosage amounts described herein refer to individual amounts administered. When more than one compound is administered, the preferred dosages correspond to the total amount of the compounds of the invention administered. The oral compositions described herein may contain from about 10% to about 95% active ingredient by weight.

The minimum inhibitory concentration for compounds of Formulae (I) and (III) are shown in the below table.

TABLE 1

| | MIC (μg/mL) | | | MIC (μg/mL) | |
|---|---|---|---|---|---|
| Pseudomonas aeruginosa PA01 | 8 | 16 | FaDu (pharynx, epithelial) | >1000 | >1000 |
| Pseudomonas aeruginosa PA14 | 16 | 32 | Hep G2 (liver, epithelial) | >1000 | >1000 |
| Escherichia coli MG1655 | 16 | 8 | HEK-293 (kidney, epithelial) | >1000 | >1000 |
| Escherichia coli AR350 | 8 | 16 | A549 (lung, epithelial) | >1000 | >1000 |
| Escherichia coli ATCC 25922 | 8 | — | | (I) | (III) |
| Escherichia coli K-12 MG1655 | 16 | — | | | |
| Salmonella enterica typhimurium | 8 | — | | | |
| LT2A TCC 19585 | | | | | |
| Salmonella enterica Enteritidis AR496 | 8 | 32 | | | |
| Acinetobacter baumannii ATCC 19606 | 16 | 8 | | | |
| Klebsiella pneumoniae AR347 | 64 | 125 | | | |
| Proteus mirabilis H14320 | 64 | 32 | | | |
| Proteus mirabilis BB2000 | 125 | 64 | | | |
| Yersinia pseudotuberculosis ATCC 6904 | 8 | 16 | | | |
| Photorhabdus australis DSM 17609 | 250 | 500 | | | |
| Burkholderia cenocepacia ATCC BAA-245 | >1000 | >1000 | | | |
| Enterobacter cloaceae ATCC 13047 | 16 | — | | | |
| Moraxella catarrhalis ATCC 25248 | 2 | — | | | |
| Shigella sonnei ATCC 25931 | 4 | — | | | |
| Shigella flexneri KLE 2512 | | — | | | |
| Vibrio vulnificus KLE δ-1125 | 8 | — | | | |
| Veillonella ratti KLE 2365 | 16 | — | | | |
| Bacteroides fragilis KLE 2244 | >128 | — | | | |
| Bacteroides stercoris KLE 2537 | >128 | — | | | |
| Photorhabdus australis DSM 17609 | >128 | — | | | |
| Staphylococcus aureus HG003 | >1000 | >1000 | | | |
| Bacillus subtilis 168 | >1000 | >1000 | | | |
| Limosilactobaccilus reuteri LTH5448 | >128 | — | | | |
| Clostridium bifermentans KLE 2329 | >128 | — | | | |
| | (I) | (III) | | | |

In general, suitable dosage ranges for intravenous (i.v.) administration of individual components are 0.001 mg to 1000 mg per kilogram body weight, 0.01 mg to 100 mg per kilogram body weight, 0.1 mg to 50 mg per kilogram body weight, and 1 mg to 10 mg per kilogram body weight. In general, suitable dosage ranges for intranasal administration of the individual components are generally from about 0.01 pg/kg body weight to 1 mg/kg body weight. In general, suppositories generally contain between about 0.01 mg to 50 mg of a compound per kilogram body weight and may comprise active ingredient in the range of 0.5% to 10% by weight. Effective doses may be extrapolated from dose-response curves derived from in vitro or animal model test systems. Such animal models and systems are well known in the art.

The invention also pertains to pharmaceutical packs or kits comprising one or more containers filled with one or more compounds to be administered in practicing the methods of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound.

The compounds described herein are useful in the treatment of infections by bacteria which are susceptible or multidrug resistant, polymyxin-resistant mutant, carbapenam-resistant bacteria or multi-drug resistant Neisseria gonorrhoeae.

Examples of Gram-negative bacteria include, but are not limited to, Escherichia coli, Pseudomonas aeruginosa, Candidatus Liberibacter, Agrobacterium tumefaciens, Moraxella catarrhalis, Citrobacter di versus, Enterobacter aerogenes, Klebsiella pneumoniae, Proteus mirabilis, Salmonella typhimurium, Neisseria meningitidis, Serratia marcescens, Shigella sonnei, Shigella boydii, Neisseria gonorrhoeae, Acinetobacter baumannii, Salmonella enteriditis, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Vibrio cholera, Morganella morganii, Edwardsiella tarda, Campylobacter jejuni, or Haemophilus influenza, Enterobacter cloacae and numerous others. Other notable groups of Gram-negative bacteria include the cyanobacteria, spirochaetes, green sulfur and green non-sulfur bacteria.

Medically relevant Gram-negative cocci include three organisms that cause a sexually transmitted disease (Neisseria gonorrhoeae), a meningitis (Neisseria meningitidis), and respiratory symptoms (Moraxella catarrhalis).

Medically relevant Gram-negative bacilli include a multitude of species. Some of them primarily cause respiratory problems (Hemophilus influenzae, Klebsiella pneumoniae, Legionella pneumophila, Pseudomonas aeruginosa), primarily urinary problems (Escherichia coli, Enterobacter cloacae), and primarily gastrointestinal problems (Helicobacter pylori, Salmonella enterica).

Gram-negative bacteria associated with nosocomial infections include Acinetobacter baumannii, which causes bacteremia, secondary meningitis, and ventilator-associated pneumonia in intensive-care units of hospital establishments. In one embodiment the compounds and compositions of the present invention are useful in the treatment of infection of one or more of the following Gram-negative bacteria: E. coli, S.enterica, Klebsiella: K. pneumoniae, K. oxytoca; Enterobacter: E. cloacae, E. aerogenes, E. agglomerans, Acinetobacter: A. calcoaceticus, A. baumannii; Pseudomonas aeruginosa, Stenotrophomonas maltophila, Providencia stuartii, Proteus, P. mirabilis, P. vulgaris.

In one embodiment, compounds of the invention or pharmaceutically acceptable salts thereof or compositions comprising the same are useful for the treatment of Pseudomonas infections including P. aeruginosa infection, for example, skin and soft tissue infections, gastrointestinal infection, urinary tract infection, pneumonia and sepsis.

In one embodiment, compounds of the invention, or pharmaceutically acceptable salts thereof, or compositions comprising the same are useful for the treatment of Acinetobacter infections including A. baumanii infection, for pneumonia, urinary tract infection and sepsis.

In one embodiment, compounds of the invention, or pharmaceutically acceptable salts thereof, or compositions comprising the same are useful for the treatment of Klebsiella infections including K. pneumoniae infection, for pneumonia, urinary tract infection, meningitis and sepsis.

In one embodiment, compounds of the invention, or pharmaceutically acceptable salts thereof, or compositions comprising the same are useful for the treatment of E. coli infection including E. coli infections, for bacteremia, cholecystitis, cholangitis, urinary tract infection, neonatal meningitis and pneumoniae.

The compounds of the invention may be prepared by growing, under controlled conditions, a strain of microorganism, Photorhabdus australis strain DSM 17609. The compound is obtained by fermentation and recovered in substantially pure form as described herein. In particular, the compounds of the invention may be produced by a strain of Photorhabdus australis strain DSM 17609 during the aerobic fermentation of suitable nutrient media under the conditions described hereinafter. The media such as those used for the production of many antimicrobial substances are suitable for use in this process for the production of the present compound.

One embodiment of the invention comprises a process suitable for producing antibiotic agents, for example, any of Formulae (I)-(XIII), by submerged aerobic fermentation of Photorhabdus australis strain DSM 17609. The compound may be recovered from the fermentation broth by resin absorption and eluted from the resin by washing with solvents of various polarities. Purification may be furthered by chromatographic separation such as reverse-phase high-performance chromatography (RP-HPLC).

Additional microorganisms capable of producing one or more compounds of the present invention include mutant species, which show advantageous properties compared with species known in the art. Such bacterial strains can be generated by mutagenesis of a parent strain. Strategies and methods of mutagenesis, procedures for screening and isolation of mutated bacterial strains, composition of media used in producing the mutant strains of the invention are known in the art.

In the preferred embodiment, cultivation of Photorhabdus australis strain DSM 17609 for the production of a compound of the invention is carried out in a nutrient medium containing readily assimilable carbon sores, nitrogen sources, inorganic salts and other organic ingredients with one or more absorbents under proper aeration conditions and mixing in a sterile environment. Compositions of nutrient media used in producing antibiotics of the invention will be described in detail in the examples. (The term “nutrient medium” as used herein describes a mixture of synthetic or naturally occurring ingredients. In general, a nutrient medium comprises a carbon source, a nitrogen source, trace elements such as inorganic salts, and optionally vitamins or other growth factors.)

In a representative fermentation and purification procedure, Photorhabdus australis DSM 17609 was grown on a Petri dish of tryptic soy agar supplemented with bromothymol blue (0.025% w/v) and triphenyltetrazolium chloride (0.004% w/v) for a period of two days at 28 degrees Celsius, having previously been stored as a freezer stock at −80 degrees Celsius. A single primary phase colony of this strain was used to inoculate a starter culture. This starter culture were 5 mL of tryptic soy broth (TSB), or any similar volume in a tube or vessel of roughly twice the total liquid volume. This starter culture was grown at 28 degrees Celsius with shaking (200 rpm) for 24 hours and subsequently used to inoculate a fermentation vessel containing TSB or Modified Grace's Medium (TNM-FH). The amount of starter culture used for the inoculation was be 1% of the final fermentation volume. Herein, we used a 2 liter non-baffled Erlenmeyer flask containing 500 mL total volume of TSB. The inoculated fermentation flask of Photorhabdus australis DSM 17609 in TSB was allowed to grow for 8-10 days at 28 degrees Celsius with shaking (200 rpm).

After this period, cells were removed by centrifugation at 8000 rcf for a period of 10 minutes, and the supernatant was retained. To the separated supernatant, 20% v/v polyaromatic adsorptive resin (styrene-divinylbenzene, AMBERLITE XAD16N, SIGMA-ALDRICH) was then added for an incubation period of 16 hours with gentle shaking, and resin was then retained. The resin was washed with roughly ten resin volumes of deionized water, and eluted with five resin volumes of acidified methanol (methanol containing 1% formic acid). This eluate was then evaporated and exchanged into acidified deionized water (water containing 1% formic acid). This was subsequently applied to a cation exchange column (180 mL of SP Sepharose fast flow resin, GE Healthcare), washed with acidic pH 50 mM ammonium acetate buffers in a step gradient, and eluted using 50 mM ammonium acetate pH 7 buffer. This eluate was then concentrated by rotary evaporator and applied to a RP-HPLC C18 column (XBridge, 250×21 mm, 5 micron) for final purification of individual peaks (compounds of Formulae I-VI), using a linear gradient of 2-30%: solvent A (water+0.1% formic acid) and solvent B (acetonitrile+0.1% formic acid) over 28 minutes at a flow rate of 15 mL/min.

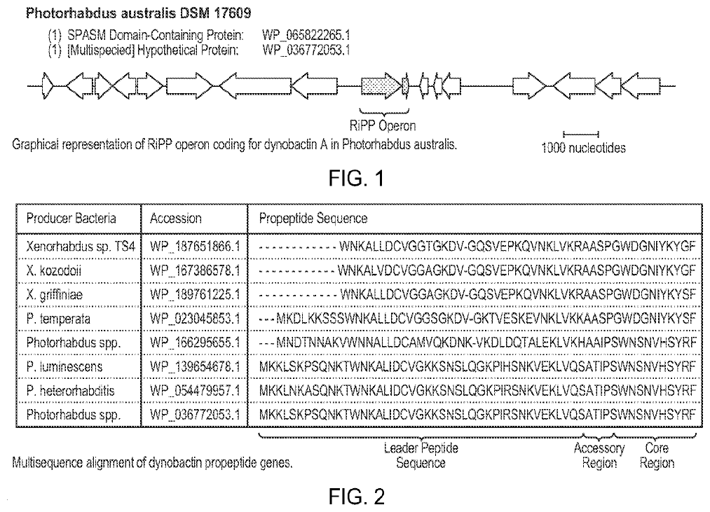

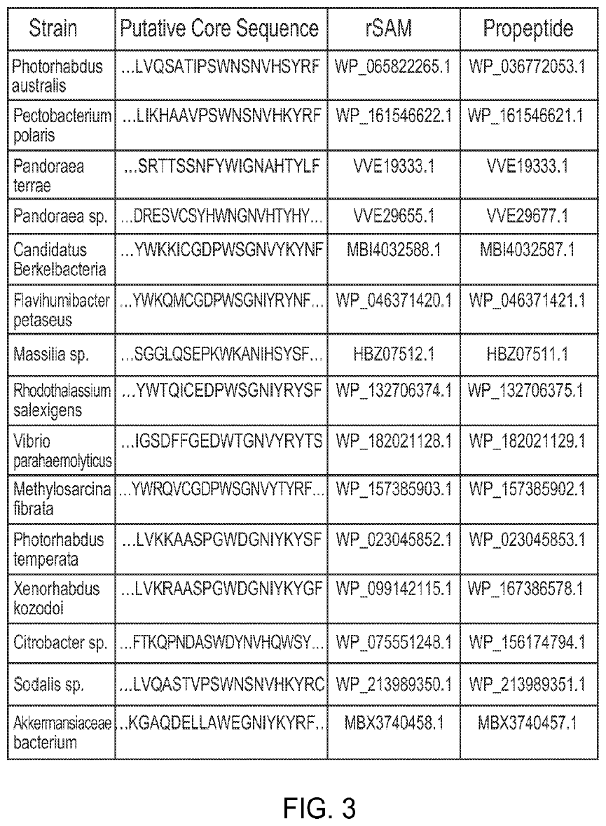

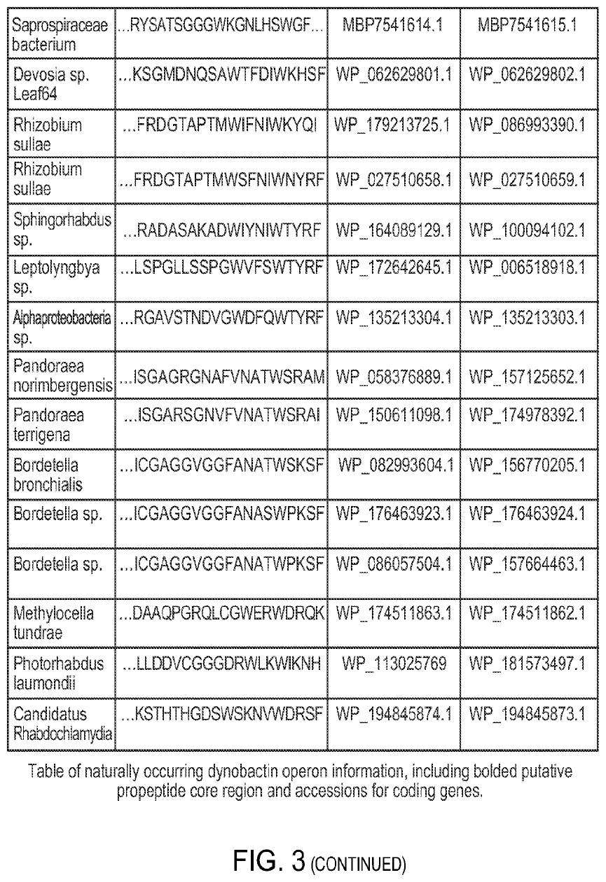

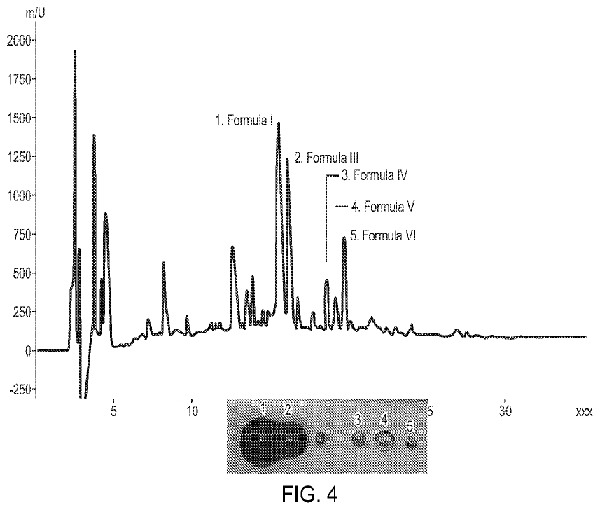

Analogs of the compounds described herein may be generated biosynthetically using straightforwardly obtained variations of the wild-type genome sequence encoding the present compounds. According to the genome sequence of Photorhabdus australis DSM 17609 (FIG. 1) and as shown in FIG. 2, there is a match between the linearized amino-acid sequence of compounds of the invention and part of a gene belonging to an operon typical for encoding RiPP (ribosomally synthesized and post-translationally-modified peptides)-type antimicrobials. The operon contains a biosynthetic gene cluster.

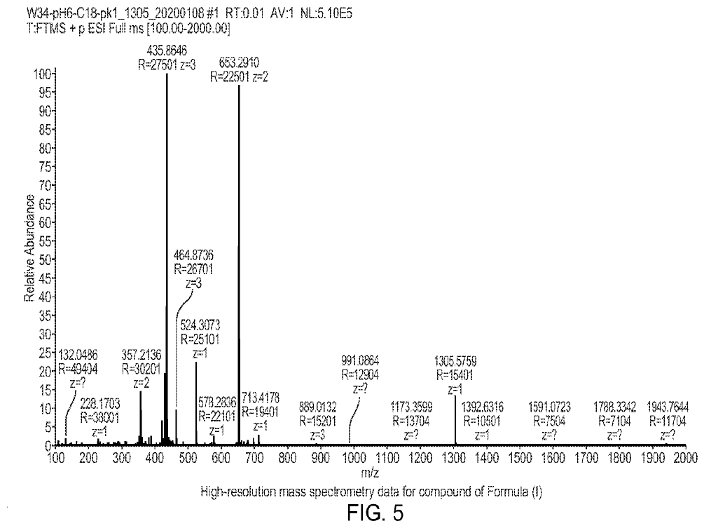

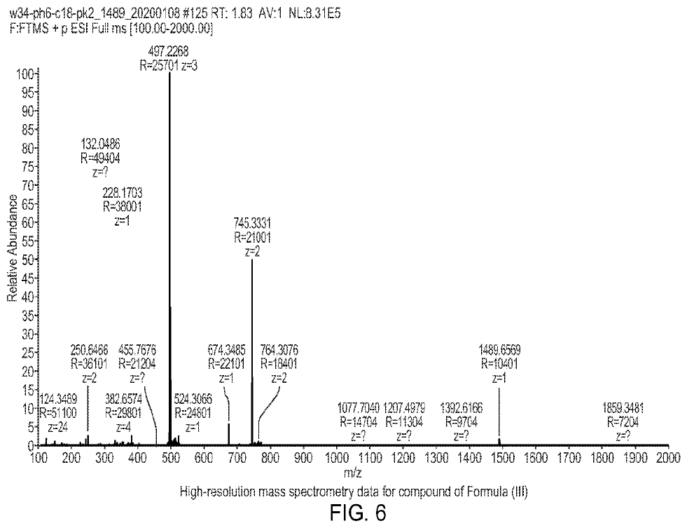

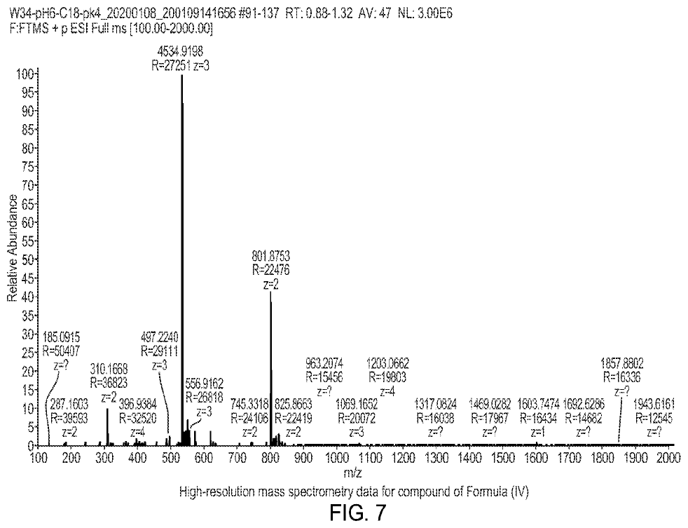

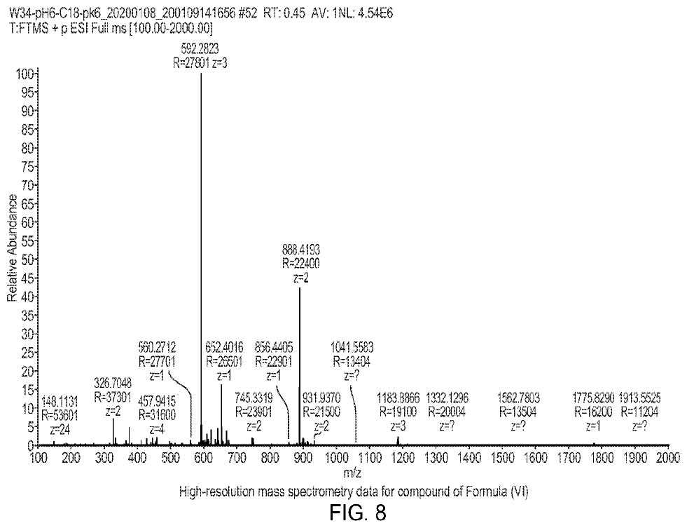

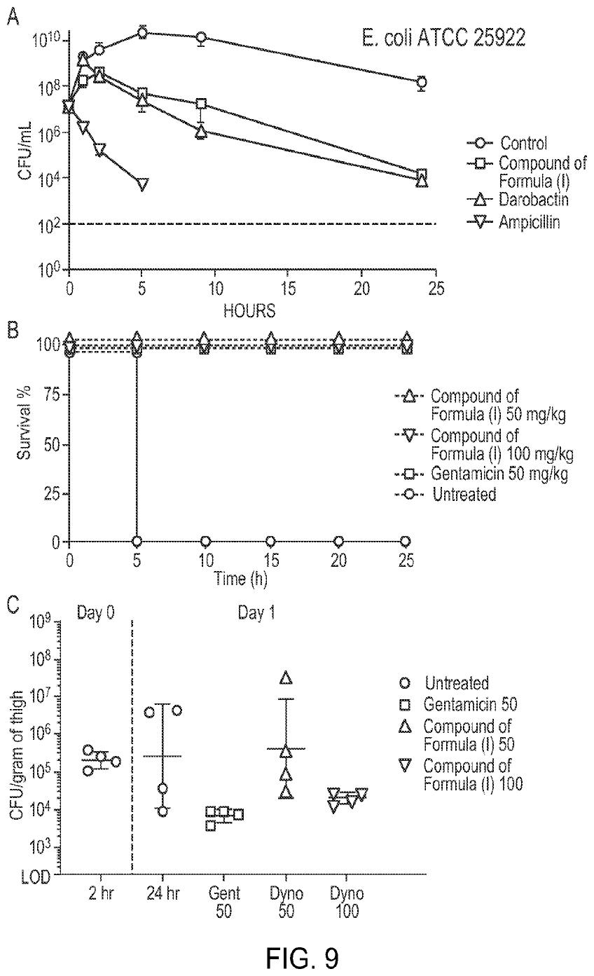

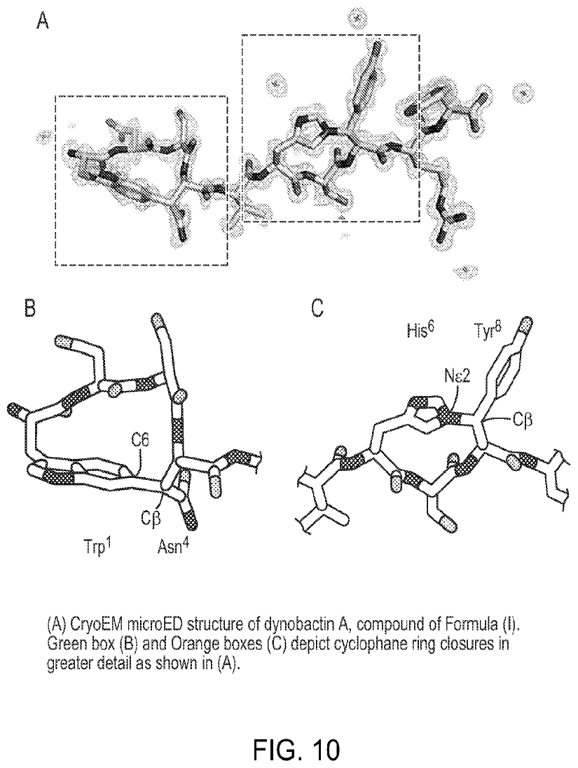

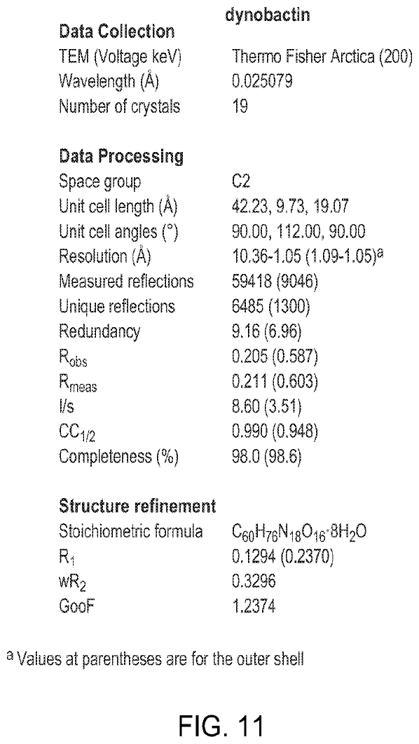

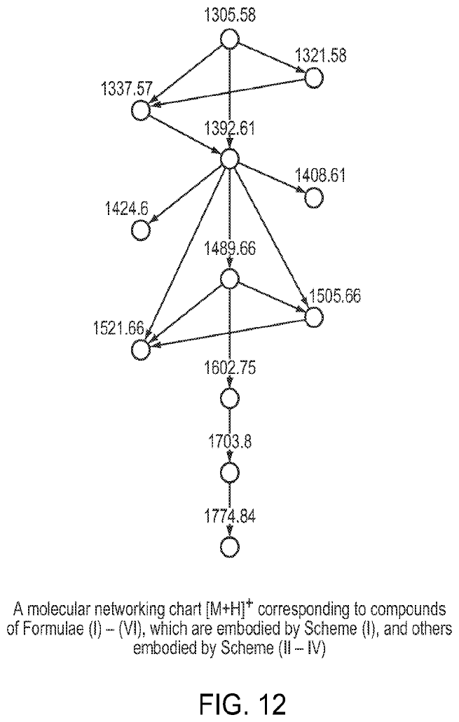

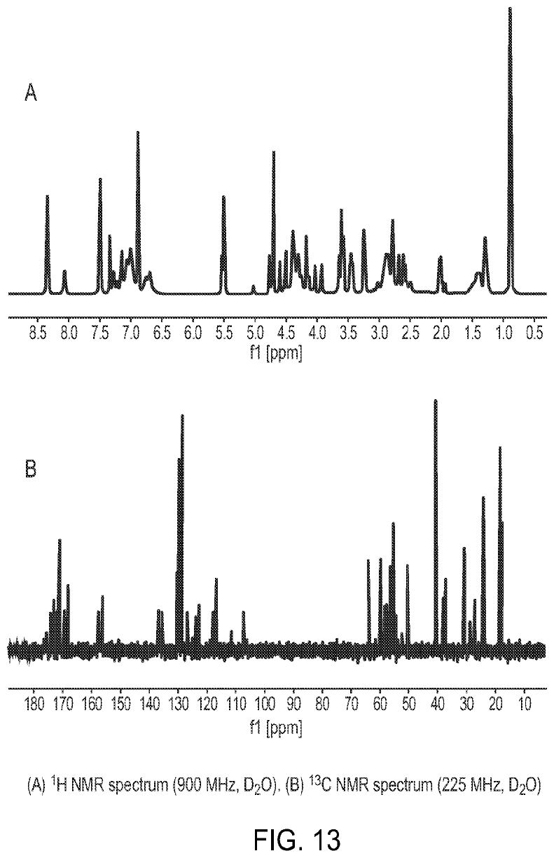

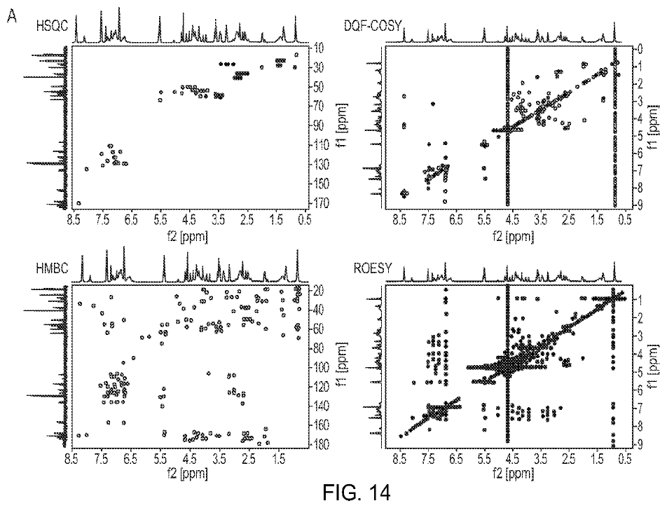

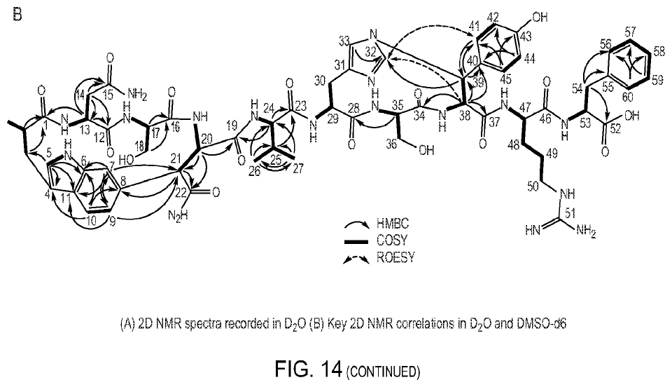

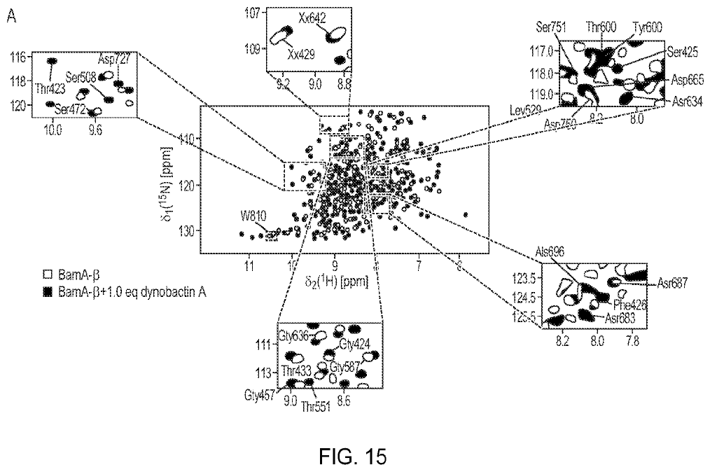

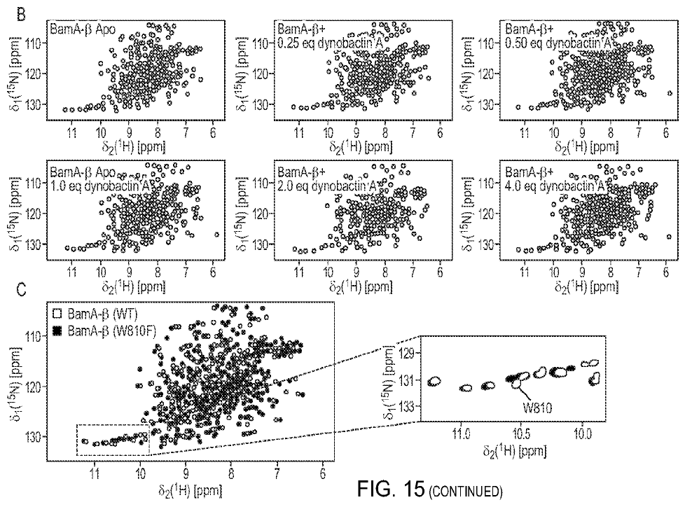

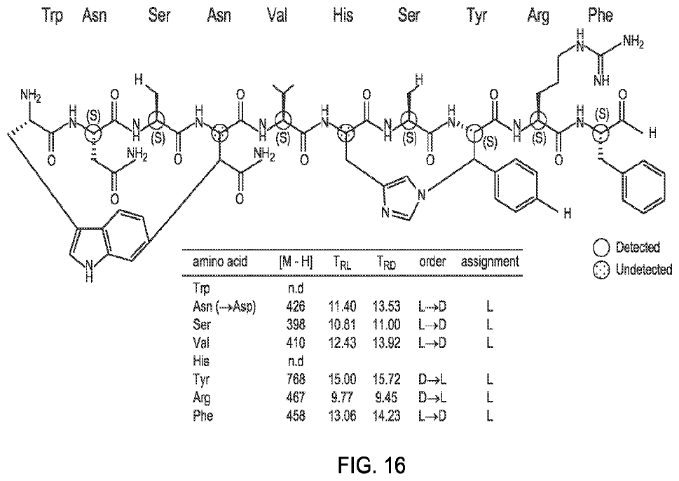

As shown in FIGS. 5-8, high-resolution mass spectrometry was used to validate the molecular weight of compounds embodied by Formulae (I), (III), (IV), and (VI). The structure of the compound embodied by Formula (I) was solved and validated by cryoelectron microscopy microcrystal electron diffraction (cryoEM microED) (FIGS. 10 and 11), nuclear magnetic resonance (NMR) (FIGS. 13-15), and Marfey's analyses (FIG. 16).

Where technically appropriate, embodiments may be combined and thus the disclosure extends to all permutations/combinations of the embodiments provided herein. The examples herein are for illustrative purposes only and they are not intended to limit the scope of the invention in any way.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A compound of any of Formulae (I)-(XIII) or Schemes (I)-(IV) or a salt, hydrate or prodrug thereof: 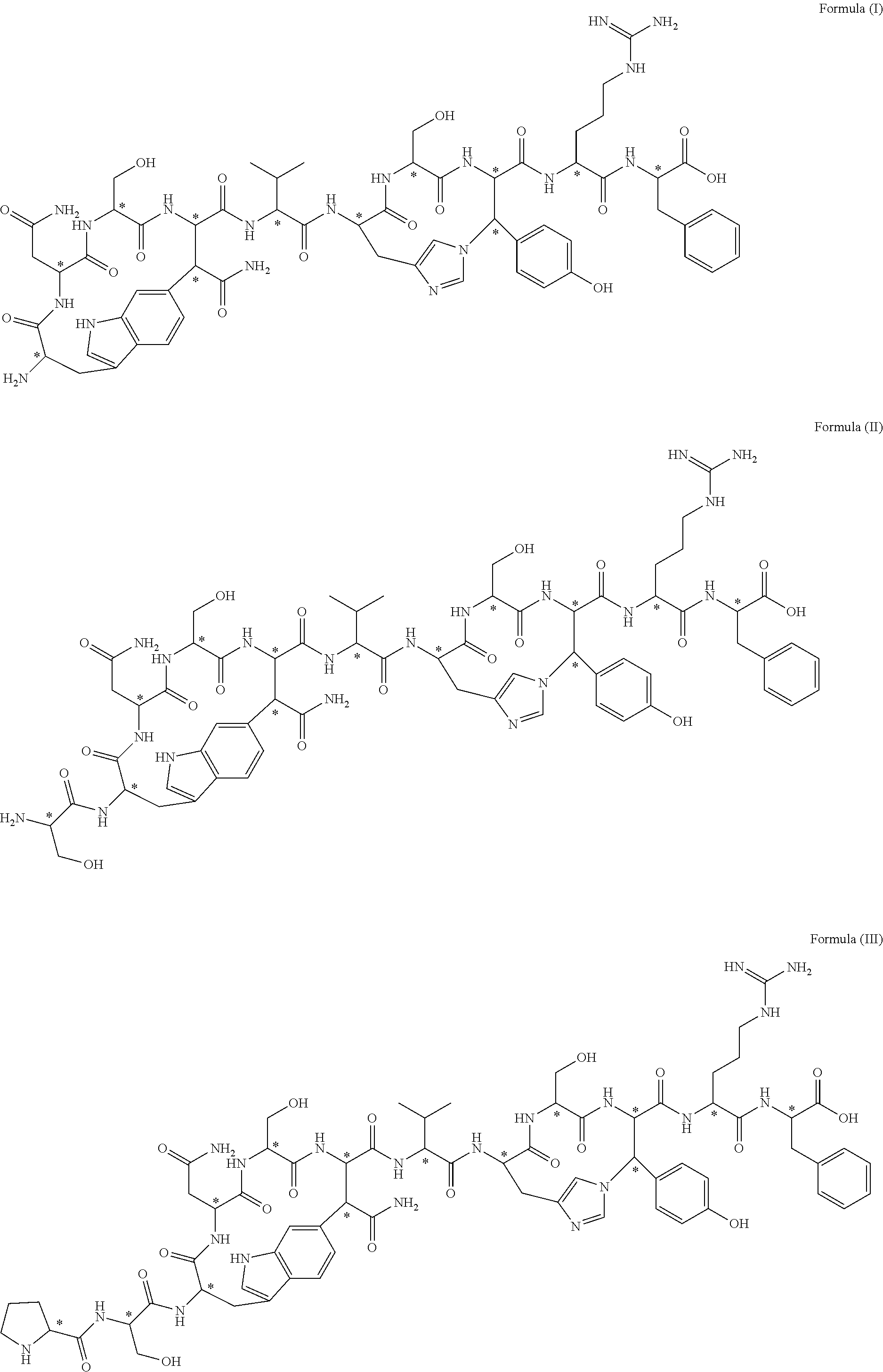 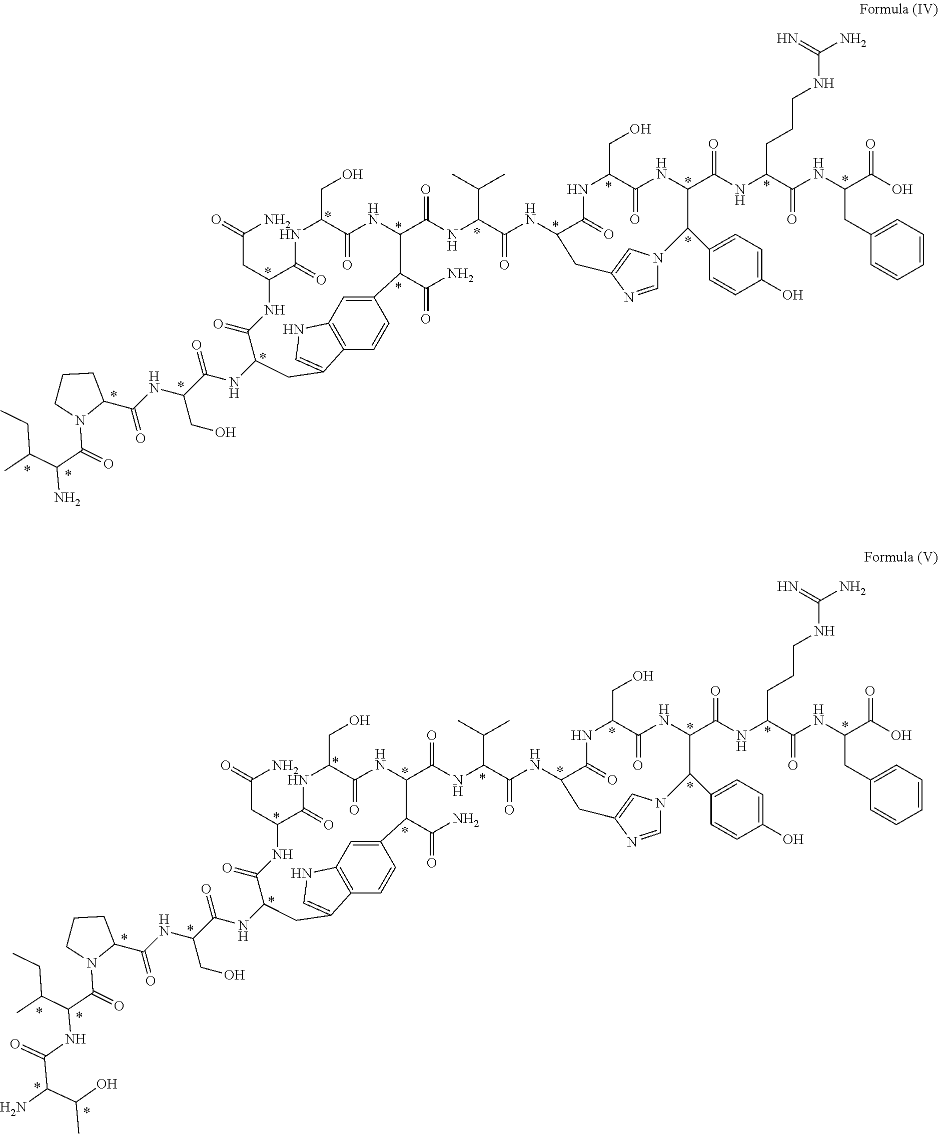 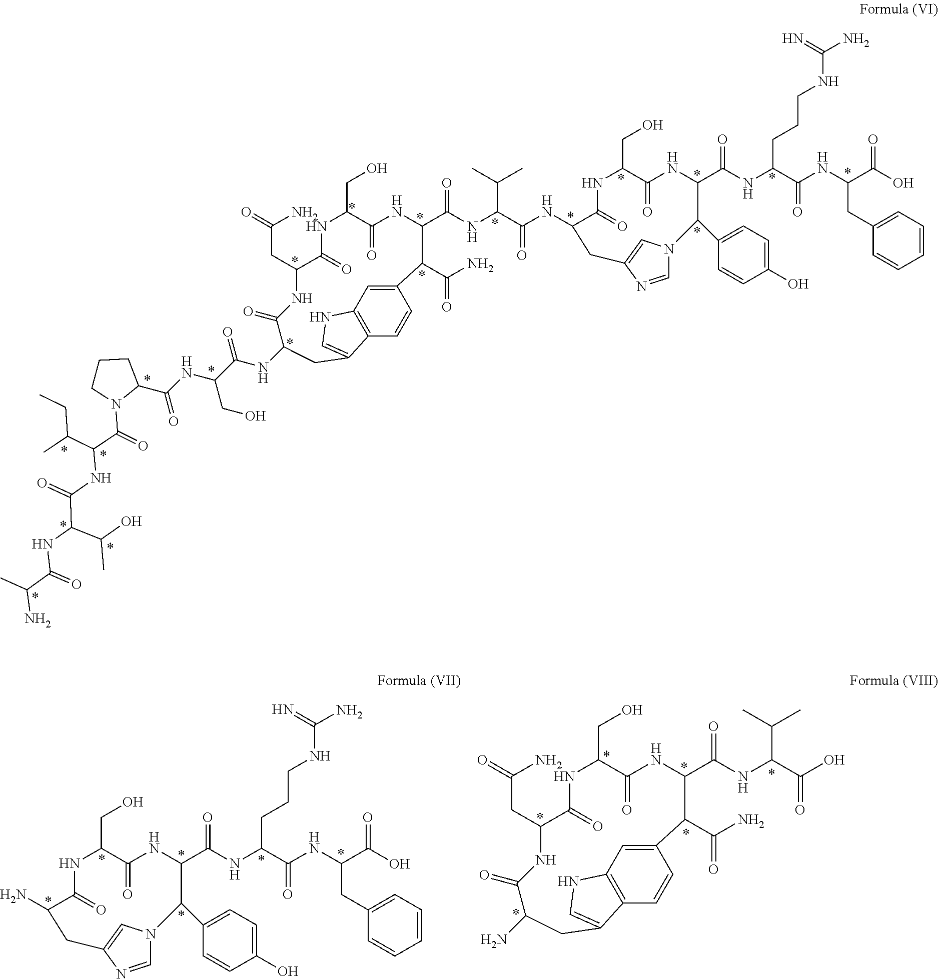 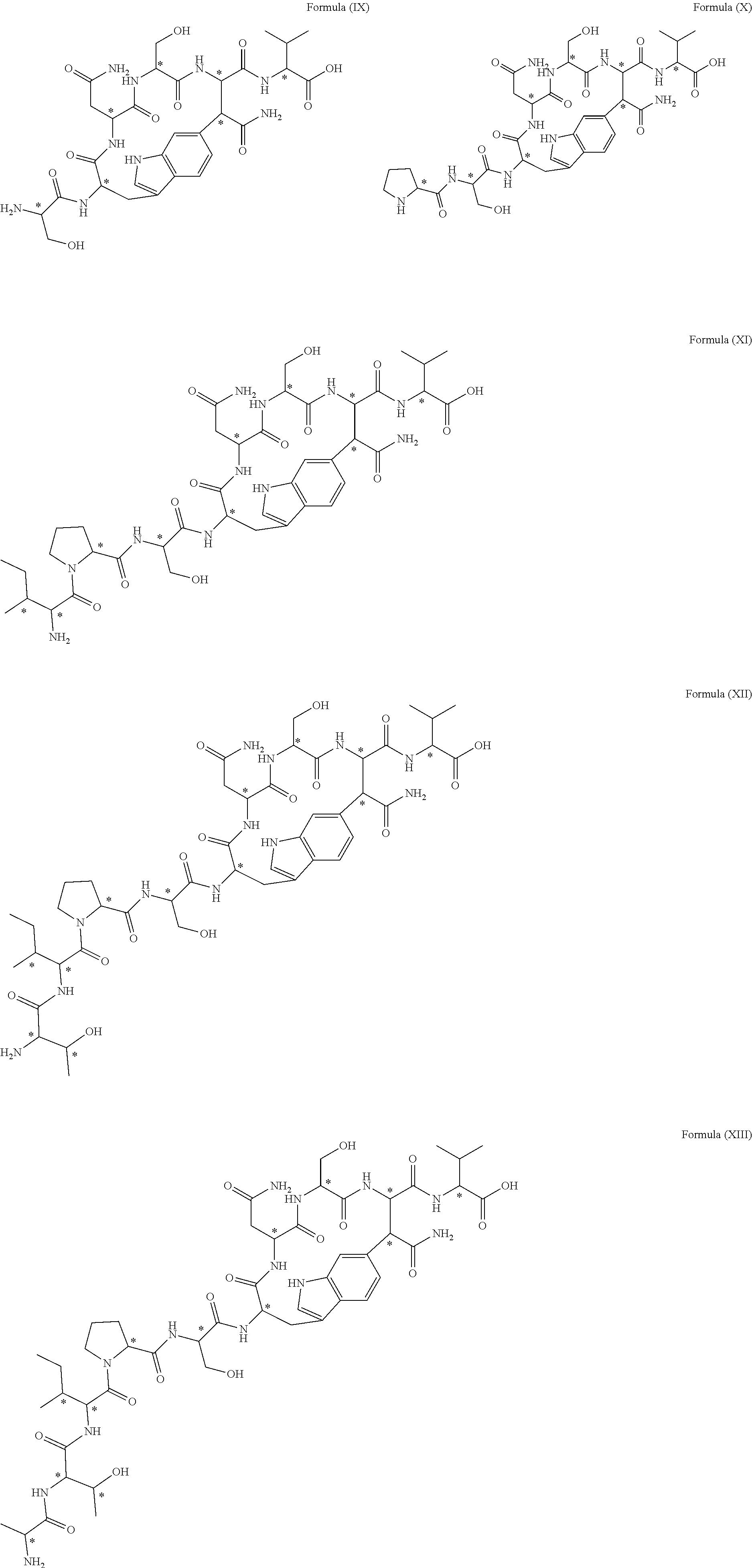 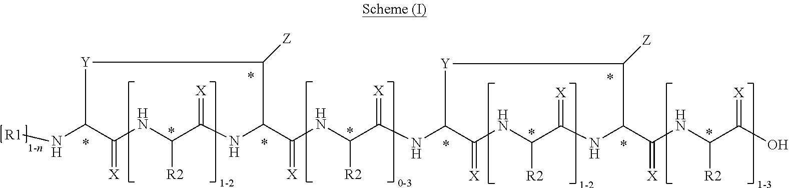 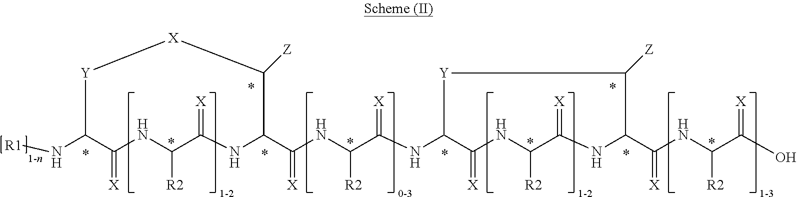 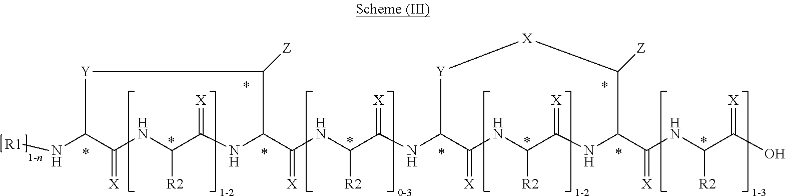 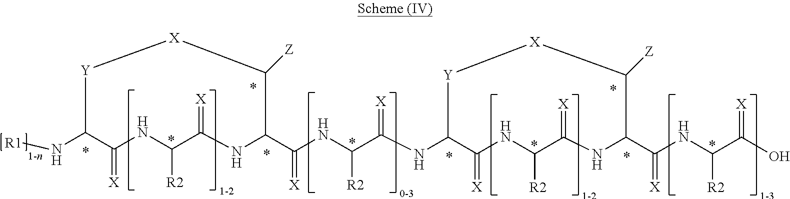
wherein, in Schemes (I)-(IV),
R1 at any position of R1 are independent from any other position of R1, and represent a hydrogen or an N-terminal extension by any number and combination of natural and/or non-natural amino acid(s),
R2 at any position of R2 are independent from any other position of R2, and indicate a side chain of a natural and/or non-natural amino acid,
X at any position of X are independent from any other position of X, and represent either an oxygen (O) or sulfur (S),
Y at any position of Y are independent from any other position of Y, and represent any aromatic amino acid sidechain (Trp, His, Phe, Tyr), which participates in macrocyclization to a neighboring β-carbon moiety, further illustrated by substructures Y.I-Y.VII,
Z at any position of Z are independent from any other position of Z, and represent a sidechain of an amino acid beginning after a β-carbon, including natural and non-natural amino acid(s) that contain a β-carbon, and
n represents a variable number of amino acids as an extension; in various embodiments, n is 1, 2, 3, 4, 5, 6, 7, 8, 9, 10, or more than 10, 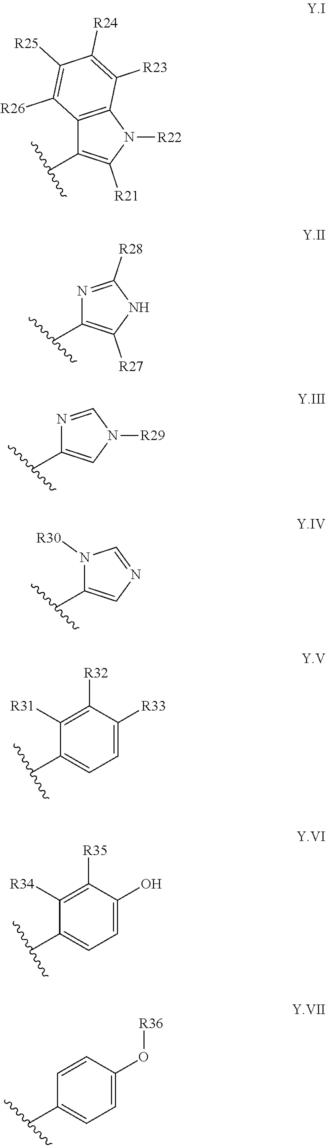
wherein, in Formula Y.I-YVII,
R21 and R36 are each independently selected from the group consisting of hydrogen, alkyl, alkenyl, alkynyl, hydroxyl, hydroxyalkyl, halogen, —CN, —O-alkyl, —C(O)-alkyl, —C(O)O-alkyl, —C(O)OH, —C(O)NH₂, —C(O)NH-alkyl, —NH₂, —NO₂, —CF₃, —NH-alkyl, —N—(alkyl)₂, —NHC(O)— alkyl and aryl, wherein said alkyl, alkenyl, alkynyl and aryl are each optionally substituted.

2. A pharmaceutical composition for treating infections in an animal caused by Gram-negative bacteria, comprising a therapeutically effective amount of the compound according to claim 1 or a pharmaceutically acceptable salt thereof.

3. The pharmaceutical composition according to claim 2, further comprising at least one pharmaceutically acceptable carrier, excipient or diluent.

4. The pharmaceutical composition according to claim 2 or 3, in a form of topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

5. The pharmaceutical composition according to any one of claims 2 to 4, further comprising at least one additional therapeutic agent.

6. The pharmaceutical composition according to any of claims 2 to 5, obtained by culturing a microorganism having an ability to produce the compound in a nutrient medium.

7. The pharmaceutical composition according to any one of claims 2 to 6, wherein the microorganism is Photorhabdus australis strain DSM 17609.

8. A method of treating, ameliorating or preventing a bacterial infection or a disease comprising administering to a subject in need thereof a therapeutically effective amount of the compound according any one of claims 1-7 or a pharmaceutically acceptable salt thereof.

9. The method according to claim 8, wherein the bacteria are Gram-negative.

10. The method according to claim 9, wherein the Gram-negative bacteria are Escherichia coli, Pseudomonas aeruginosa, Candidatus Liberibacter, Agrobacterium tumefaciens, Acinetobactor baumannii, Moraxella catarrhalis, Citrobacter di versus, Enterobacter aerogenes, Klebsiella pneumoniae, Proteus mirabilis, Salmonella typhimurium, Neisseria meningitidis, Serratia marcescens, Shigella sonnei, Shigella boydii, Neisseria gonorrhoeae, Acinetobacter baumannii, Salmonella enteriditis, Fusobacterium nucleatum, Veillonella parvula, Actinobacillus actinomycetemcomitans, Aggregatibacter actinomycetemcomitans, Porphyromonas gingiva/is, Helicobacter pylori, Francisella tularensis, Yersinia pestis, Vibrio cholera, Morganella morganii, Edwardsiella tarda, Campylobacter jejuni, or Haemophilus influenza, Enterobacter cloacae, or other Gram-negative pathogens.

11. The method according to any one of claims 8-10, wherein the bacterial are susceptible or multidrug-resistant.

12. The method according to any one of claims 8-11, wherein the bacteria are multidrug-resistant.

13. The method according to any one of claims 8-12, wherein the bacterial are polymyxin-resistant.

14. The method according to any one of claims 8-13, wherein the bacteria are carbapenam-resistant bacteria or multi-drug resistant Neisseria gonorrhoeae.

15. The method according to any of claims 8-14, wherein the bacterial infection is a respiratory infection, a skin or skin structure infection, urinary infection, an intra-abdominal infection, a blood stream infection, a gastrointestinal infection.

16. The method according to any of claims 8-15, wherein the disease is selected from the group consisting of skin inflammatory diseases, inflammatory bowel disease (IBD), ulcerative colitis, Crohn's disease, and Celiac disease.

17. The method according to any of claims 8-16, wherein the administering step comprises topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

18. A composition comprising the compound represented by any one of Formulae (I)-(XIII) or Schemes (I)-(IV) according to claim 1 or a salt hydrate or prodrug thereof and a carrier.

19. The composition according to claim 18, wherein the carrier is a pharmaceutically acceptable carrier.

20. The composition according to claim 18, wherein the carrier is a agriculturally acceptable carrier.

21. The pharmaceutical composition according to any one of claims 18 to 20, in a form of topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

22. The pharmaceutical composition according to any one of claims 18 to 21, further comprising at least one additional therapeutic agent.

23. The pharmaceutical composition according to any of claims 18 to 22, obtained by culturing a microorganism having an ability to produce the compound in a nutrient medium.

24. The pharmaceutical composition according to any one of claims 18 to 23, wherein the microorganism is Photorhabdus australis strain DSM 17609.

25. A composition for combatting, controlling or inhibiting a pest, comprising a pesticidally effective amount of the compound according to claim 1 or a salt thereof.

26. The composition according to claim 25, further comprising at least one agriculturally acceptable carrier, excipient or diluent.

27. The composition according to claim 25 or 26, in a form of topical administration, systemic administration, parenteral administration, subcutaneous administration, or transdermal administration, rectal administration, oral administration, intravaginal administration, intranasal administration, intrabronchial administration, intraocular administration, intra-aural administration, intravenous administration, intramuscular administration, or intraperitoneal administration.

28. The composition according to any one of claims 25 to 27, further comprising at least one additional therapeutic agent.

29. The composition according to any of claims 25 to 28, obtained by culturing a microorganism having an ability to produce the compound in a nutrient medium.

30. The pharmaceutical composition according to any one of claims 25 to 29, wherein the microorganism is Photorhabdus australis strain DSM 17609.

31. A method of combatting, controlling or inhibiting a pest comprising exposing the pest to a pesticidally effective amount of any one of the compounds represented by any one of Formulae (I)-(XIII) or Schemes (I)-(IV) according to claim 1 or a salt, hydrate or prodrug thereof.

32. The method according to claim 31, wherein the bacteria are Gram-negative.

33. The method according to claim 32, wherein the Gram-negative bacteria can be Escherichia coli, Pseudomonas aeruginosa, Candidatus Liberibacter, Agrobacterium tumefaciens, Acinetobactor baumannii, Moraxella catarrhalis, Citrobacter di versus, Enterobacter aerogenes, Klebsiella pneumoniae, Proteus mirabilis, Salmonella typhimurium, Neisseria meningitidis, Serratia marcescens, Shigella sonnei, Shigella boydii, Neisseria gonorrhoeae, Acinetobacter baumannii, Salmonella enteriditis, Fusobacterium nucleatum, Veillonella parvula, Actinobacillus actinomycetemcomitans, Aggregatibacter actinomycetemcomitans, Porphyromonas gingivalis, Helicobacter pylori, Francisella tularensis, Yersinia pestis, Vibrio cholera, Morganella morganii, Edwardsiella tarda, Campylobacter jejuni, or Haemophilus influenza, Enterobacter cloacae, or other Gram-negative pathogens.


SEQUENCE LISTING (13 sequences)

SEQ ID NO: 1 - protein, 10 aa, Photorhabdus australis
WNSNVHSYRF

SEQ ID NO: 2 - protein, 11 aa, Photorhabdus australis
SWNSNVHSYRF

SEQ ID NO: 3 - protein, 12 aa, Photorhabdus australis
PSWNSNVHSYRF

SEQ ID NO: 4 - protein, 13 aa, Photorhabdus australis
IPSWNSNVHSYRF

SEQ ID NO: 5 - protein, 14 aa, Photorhabdus australis
TIPSWNSNVHSYRF

SEQ ID NO: 6 - protein, 15 aa, Photorhabdus australis
ATIPSWNSNVHSYRF

SEQ ID NO: 7 - protein, 5 aa, Photorhabdus australis
HSYRF

SEQ ID NO: 8 - protein, 5 aa, Photorhabdus australis
WNSNV

SEQ ID NO: 9 - protein, 6 aa, Photorhabdus australis
SWNSNV

SEQ ID NO: 10 - protein, 7 aa, Photorhabdus australis
PSWNSNV

SEQ ID NO: 11 - protein, 8 aa, Photorhabdus australis
IPSWNSNV

SEQ ID NO: 12 - protein, 9 aa, Photorhabdus australis
TIPSWNSNV

SEQ ID NO: 13 - protein, 10 aa, Photorhabdus australis
ATIPSWNSNV